(19) United States
(12) Patent Application Publication
(10) Pub. No.: US 2021/0128675 A1
(43) Pub. Date: May 6, 2021

(54) LHRH-PACLITAXEL CONJUGATES AND METHODS OF USE

(71) Applicant: Worcester Polytechnic Institute, Worcester, MA (US)

(72) Inventors: Winston O. Soboyejo, Northborough, MA (US); John D. Obayemi, Shrewsbury, MA (US); Ali A. Salifu, Worcester, MA (US); Vanessa O. Uzonwanne, Worcester, MA (US)

(21) Appl. No.: 17/085,957

(22) Filed: Oct. 30, 2020

Related U.S. Application Data
(60) Provisional application No. 62/928,549, filed on Oct. 31, 2019.

(51) Int. Cl. A61K 38/09; A61K 31/337; A61K 47/64; A61K 47/55; A61K 47/54; A61P 35/00; A61K 9/50; A61K 9/00

(52) U.S. Cl. CPC A61K 38/09; A61K 31/337; A61K 47/64; A61K 47/55; A61K 9/0019; A61K 47/54; A61P 35/00; A61K 9/5031; A61K 47/545

(57) ABSTRACT

Aspects of the disclosure relate to compositions comprising Luteinizing Hormone Releasing Hormone (LHRH) or an analog of LHRH conjugated to paclitaxel, and methods of treatment of cancer, for example, triple negative breast cancer, using such compositions.

### RELATED APPLICATIONS

This application claims priority to and the benefit of U.S. Provisional Application No. 62/928,549, filed Oct. 31, 2019, the disclosure of which is hereby incorporated by reference in its entirety for all purposes.

### REFERENCE TO SEQUENCE LISTING

The instant application contains a Sequence Listing which has been submitted electronically in ASCII format and is hereby incorporated by reference in its entirety. Said ASCII copy, created on Oct. 29, 2020, is named 110697-015501US_SL.txt and is 1,769 bytes in size.

### TECHNICAL FIELD

The disclosure relates generally to conjugate drugs, compositions thereof and methods for use thereof for treating cancer. In particular, the instant disclosure relates to LHRH conjugates for the treatment of triple negative breast cancer.

### BACKGROUND

Breast cancer is the most commonly diagnosed cancer and the second cause of death in women. In general, breast tumors are intrinsically heterogeneous in nature. This makes them difficult to detect and treat. Statistics has shown that about 75-80% of breast cancers are hormone receptor-positive. These overexpressed receptors can be estrogen and/or progesterone receptors. However, about 10-15% of breast cancers (for example, Triple negative breast cancer (TNBC)) do not express either estrogen or progesterone receptors, or the human epidermal growth factor receptor 2 gene (HER2). TNBCs account for 10-17% of all breast carcinomas. They also exhibit distinctive clinical features and are more common in younger patients and African American/African women.

Conventional treatments are limited by poor therapeutic response and aggravated side effects. In view of this problems, effective methods for treating patient suffering from TNBC are needed.

### SUMMARY

The present disclosure provides conjugates of LHRH or LHRH analog and paclitaxel active agent. The present disclosure further provides pharmaceutical compositions comprising such conjugates as well as methods of treatment of cancer using such conjugates. In some embodiments, the conjugates can be provided as a delayed release composition loaded in microspheres.

In some aspects, the present disclosure provides a conjugate comprising a Luteinizing Hormone Releasing Hormone (LHRH) or an analog of LHRH conjugated to paclitaxel active agent. In some embodiments, the analog of LHRH is D-Lys6 LHRH. In some embodiments, the paclitaxel active agent is conjugated at the epsilon (ε) amino side chain of the LHRH or the analog of LHRH. In some embodiments, a hydrophilic linker conjugates paclitaxel active agent to the LHRH or the LHRH analog. Such linker can be N-hydroxysuccinimide, 1-ethyl-3-(3-dimethylaminopropyl)carbodiimide hydrochloride (EDC) or combinations thereof.

In some aspects, the present disclosure provides a pharmaceutical composition comprising (a) an effective amount of a conjugate comprising a Luteinizing Hormone Releasing Hormone (LHRH) or an analog of LHRH conjugated to paclitaxel active agent, and (b) a physiologically acceptable carrier. In some embodiments, the analog of LHRH is D-Lys6 LHRH. In some embodiments, the paclitaxel active agent is conjugated at the epsilon (c) amino side chain of the LHRH or the analog of LHRH. In some embodiments, a hydrophilic linker conjugates paclitaxel active agent to the LHRH or the LHRH analog. Such linker can be N-hydroxysuccinimide, 1-ethyl-3-(3-dimethylaminopropyl)carbodiimide hydrochloride (EDC) or combinations thereof. In some embodiments, the pharmaceutical composition comprises microspheres loaded with the conjugate. In some embodiments, the microspheres are poly lactic-co-glycolic acid-polyethylene glycol (PLGA-PEG) polymer microspheres. In some embodiments, the pharmaceutical composition is formulated for intravenous injection.

In some aspects, the present disclosure provides a method for treating breast cancer, comprising: administering to a subject in need thereof an effective amount of a pharmaceutical composition comprising a conjugate of a Luteinizing Hormone Releasing Hormone (LHRH) or an analog of LHRH conjugated to paclitaxel active agent, and a physiologically acceptable carrier. In some embodiments, the paclitaxel active agent is conjugated at the epsilon (c) amino side chain of the LHRH or the analog of LHRH. In some embodiments, a hydrophilic linker conjugates paclitaxel active agent to the LHRH or the LHRH analog. Such linker can be N-hydroxysuccinimide, 1-ethyl-3-(3-dimethylaminopropyl)carbodiimide hydrochloride (EDC) or combinations thereof. In some embodiments, the pharmaceutical composition comprises poly lactic-co-glycolic acid-polyethylene glycol (PLGA_PEG) polymer microspheres loaded with the conjugate. In some embodiments, the pharmaceutical composition is formulated for an intravenous injection. In some embodiments, the pharmaceutical composition is administered to a subject suffering from triple negative breast cancer. In some embodiments, the method comprises administering the pharmaceutical composition intravenously and subsequently injecting polymer microspheres loaded with the conjugate in proximity of tumor.

Some aspects of the present disclosure relate to a conjugate comprising Luteinizing Hormone Releasing Hormone (LHRH) analog D-Lys6 LHRH conjugated to paclitaxel, wherein the paclitaxel is conjugated at the epsilon (c) amino side chain of the D-Lys6 LHRH moiety. In some embodiments, the conjugate further comprising a hydrophilic linker to conjugate paclitaxel to the LHRH analog. In some embodiments, the linker is N-hydroxysuccinimide.

Some aspects of the present disclosure relate to a composition comprising an effective amount of a conjugate comprising Luteinizing Hormone Releasing Hormone (LHRH) analog D-Lys6 LHRH conjugated to paclitaxel, wherein the paclitaxel is conjugated at the epsilon (c) amino side chain of the D-Lys6 LHRH moiety. In some embodiments, the conjugate further comprising a hydrophilic linker to conjugate paclitaxel to the LHRH analog. In some embodiments, the linker is N-hydroxysuccinimide.

Some aspects of the present disclosure relate to methods for treating triple negative breast cancer, comprising: administering to a subject in need thereof an effective amount of a composition comprising Luteinizing Hormone Releasing Hormone (LHRH) analog D-Lys6 LHRH conjugated to paclitaxel, wherein the subject in need thereof has triple negative breast cancer.

In some aspects, the present disclosure provides methods for preparing a conjugate comprising conjugating an LHRH or an analog of LHRH conjugated to paclitaxel. In some embodiments, the LHRH or its analog are conjugated to paclitaxel in the presence of comprises N-hydroxysuccinimide (NHS), 1-ethyl-3-(3-dimethylaminopropyl)carbodiimide hydrochloride (EDC) or combinations thereof.

In some aspects, the present disclosure provides a use of a conjugate comprising a Luteinizing Hormone Releasing Hormone (LHRH) or an analog of LHRH conjugated to paclitaxel active agent in preparing a pharmaceutical composition for treating cancer, in particular triple negative breast cancer.

In some aspects, the present disclosure provides a use of a conjugate comprising a Luteinizing Hormone Releasing Hormone (LHRH) or an analog of LHRH conjugated to paclitaxel active agent for treating cancer, in particular triple negative breast cancer.

### BRIEF DESCRIPTION OF THE DRAWINGS

Aspects of the present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 shows the structure of paclitaxel (PTX).

FIG. 2A shows FTIR spectra of LHRH-conjugated PTX drug.

FIG. 2B shows LC-MS spectra of LHRH-PTX drug.

FIG. 3A shows percentage alamar blue reduction for breast cancer cells.

FIG. 3B shows percentage cell growth inhibition of breast cancer cells (10⁴ cells/well) coincubated with 15 μM, 25 μM, and 30 μM of LHRH-conjugated PTX drug in the presence of control drug for the period of 72 h. The coincubation of LHRH decreased the cytoxicity of LHRH-PTX. The data presented are the average of three independent experiments. (n=3, *P<0.05).

FIG. 3C shows percentage alamar blue reduction for knocked down LHRH receptors of breast cancer cells (104 cells/well) co-incubated with 5 μM of DMSO, LHRH, paclitaxel, and LHRH-conjugated PTX drugs for the period of 72 h.

FIG. 3D shows confocal fluorescence images showing cellular uptake and cytotoxicity comparison of MDA-MB-231 cells 6 hours after their incubation with 30 μM of PTX or LHRH-PTX (arrows indicate the structural changes in the nuclei structure, actin cytoskeleton structure and vinculin structure).

FIG. 4 shows the mean volume of the induced xenograft tumor progression just before the various staged of therapy.

FIG. 5 shows anti-tumor activity and tumor shrinkages of induced subcutaneous xenografts tumor athymic nude mice bearing triple negative breast cancer treated with two IV injections of LHRH-PTX, PTX and DMSO for 14-day study.

FIG. 6 shows anti-tumor activity and tumor shrinkages of induced subcutaneous xenografts tumor athymic nude mice bearing triple negative breast cancer treated with two IV injections of LHRH-PTX, PTX and DMSO for 21-day study.

FIG. 7 shows anti-tumor activity and tumor shrinkages of induced subcutaneous xenografts tumor athymic nude mice bearing triple negative breast cancer treated with two IV injections of LHRH-PTX, PTX and DMSO for 28-day study.

FIG. 8A shows the summary of measured pull-off force/adhesion forces for drug-coated AFM tip to triple negative breast tumor at early stage, mid stage and late stage.

FIGS. 8B-8D show immunofluorescence staining of expressed LHRH receptors on early stage (FIG. 8B), mid stage (FIG. 8C) and late stage (FIG. 8D) triple negative breast cancer tissue.

FIG. 9 shows the change in the body weight of xenograft tumor-bearing mice treated with 10 mg/kg of conjugated and unconjugated PTX drugs in the presence of control.

FIG. 10 shows histopathological examination of tumor tissues and organs in MDA-MB 231 induced xenograft breast tumor model mice after treatment with LRH-conjugated and unconjugated PTX drugs.

FIG. 11 shows the outline images of tumor shrinkages of induced subcutaneous xenografts tumor of athymic nude mice bearing triple negative breast cancer treated with two IV injections of LHRH-PTX, PTX and DMSO for the Day-14 treatment group.

FIG. 12 shows the outline images of tumor shrinkages of induced subcutaneous xenografts tumor of athymic nude mice bearing triple negative breast cancer treated with two IV injections of LHRH-PTX, PTX and DMSO for the Day-21 treatment group.

FIG. 13 shows the outline images of tumor shrinkages of induced subcutaneous xenografts tumor of athymic nude mice bearing triple negative breast cancer treated with two IV injections of LHRH-PTX, PTX and DMSO for the Day-28 treatment group.

FIG. 14A shows confocal fluorescence images showing the expression of LHRH receptors of non-tumorigenic epithelial breast cell line (MCF 10 A).

FIG. 14B shows confocal fluorescence images showing the expression of LHRH receptors of triple negative breast cancer cells (MDA-MB 231).

FIG. 14C shows confocal fluorescence images showing the expression of LHRH receptors of blocked LHRH antibody receptors on triple negative breast tissue.

FIG. 14D shows confocal fluorescence images showing the expression of LHRH receptors of stained LHRH triple negative breast tissue at 40× magnification.

FIG. 14E shows quantified fluorescence LHRH receptors in cells and tissue of TNBS.

FIG. 14F shows detection of LHRH-R knockdown by RT-qPCR.

FIG. 15 shows representative TEM micrographs showing the morphologies and ultrastructures of tumor tissue/cells from MDA-MB 231 induced xenograft breast tumor model mice after treatment with PTX, LHRH-PTX.

FIGS. 16A-16C show SEM images of PLGA-PEG-PTX, PLGA-PEG-LHRH-PTX, PLGA-PEG microspheres.

FIG. 16D shows mean particle size distributions of drug-loaded and control PLGA-PEG microspheres.

FIG. 17A shows FTIR spectra of the synthesized drug-loaded PLGA-PEG microspheres and control (PLGA-PEG) microspheres.

FIG. 17B shows a representative 1HNMR spectrum for drug-loaded PLGA-PEG microspheres.

FIG. 18A shows TGA curves of control PLGA-PEG microspheres and drug-loaded PLGA-PEG microspheres.

FIG. 18B shows DSC thermographs of freeze-dried drug-loaded and control PLGA-PEG microspheres.

FIGS. 19A-19B show in vitro release profile of PLGA-PEG-PTX and PLGA-PEG-LHRH-PTX drug-loaded microspheres at 37° C., 41° C. and 44° C., respectively. In all cases (n=3, ^{@}p>0.05 vs. control);

FIG. 20 shows a plot of Gibb's free energy versus temperature for various drug-loaded PLGA-PEG formulations.

FIG. 21 shows SEM images of surfaces of drug-loaded PLGA-PEG microspheres after 57 days exposure to phosphate buffer saline at pH 7.4 and cross-sections, with different magnification.

FIG. 22A shows percentage alamar blue reduction for cells only (MDA-MB-231 cells), drug-loaded and control PLGA-PEG microspheres after 6, 24, 48, 72 and 96 h post-treatment [*p<0.05 (n=4)].

FIG. 22B shows percentage cell growth inhibition for drug-loaded and control PLGA-PEG microspheres after 6, 24, 48, 72 and 96 h′ post-treatment [*p<0.05 (n=4)], respectively.

FIG. 23A shows cell viability study of MDA-MB-231 cells showing the effect of the treatment time when incubated with drug-loaded and unloaded PLGA-PEG microspheres after for a period of 240 h with MDA-MB-231 cells acting as a control.

FIG. 23B shows representative confocal images of MDA MB-231 cells after 5 h incubation with respective drug-loaded PLGA-PEG microspheres at 37° C.

FIG. 24A shows body weight variation of subcutaneous xenograft tumor-bearing mice treated with drug-loaded microparticles in the presence of control (n=5, \hat{ }p<0.05).

FIG. 24B shows Kaplan Meier survival curves (N=30) showing the effect of all treatment groups on the survival rate of mice.

FIGS. 25A-25D show representative immunofluorescence images of LHRH receptors expressed on the tumor (FIG. 25A), and lungs of mice (FIG. 25B) treated with a control microspheres (PLGA-PEG) and their corresponding H&E stain showing metastasis in the tumor (FIG. 25C) and lungs (FIG. 25D).

FIGS. 26A-26B show optical images of mice lungs treated with PLGA-PEG-PTX and PLGA-PEG-LHRH-PTX, respectively.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

It is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, all numerical values or numerical ranges include integers within such ranges and fractions of the values or the integers within ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

Unless defined otherwise, all technical and scientific terms used herein have the same meaning as commonly understood by one of ordinary skill in the art to which this disclosure belongs. Any methods and materials similar or equivalent to those described herein can be used in the practice or testing of the disclosure.

Aspects of the present disclosure relates generally to compositions and methods for treating patients diagnosed with cancer. LHRH receptors (LIR-R) have been shown to be expressed on over 50% of human breast cancer specimens in a non-selected patient cohort characterized by TNBC (Engel J B et al., Mol Pharm. 2007, 4: 652-658 and Fekete M. et al., J Clin Lab Anal. 1989, 3: 137-147). It was also shown that the LHRH receptors are overexpressed in human breast, ovarian and prostate cancer cells, but are below the detection limits of PCR in normal human organs (lung, liver, kidneys, spleen, muscle, heart, thymus) (Dharap et al., 2003, Pharm. Res. 20(6), 89-896). In some embodiments, the present disclosure provides methods of treatment of cancer where the cancer cells express one or more receptors that bind to LHRH or an LHRH analog, in particular, triple negative breast cancer (TNBC). In some embodiments, the compositions described herein have a Luteinizing hormone-releasing hormone (LHRH) receptors targeting moiety conjugated to an active agent against cancer. In some embodiments, the active agent is paclitaxel (PTX) drug.

### LHRH-Conjugated Paclitaxel and LHRH-Conjugated Drugs

Some aspects of the disclosure relate to drugs conjugated to LHRH, LHRH analog, peptide comprising LHRH or peptide comprising LHRH analog, methods of making the conjugated drugs, and method treating cancers, such as TNBC, using the conjugated drug.

In some embodiments, the LHRH is a decapeptide consisting of the amino acid sequence of SEQ ID NO: 1 (Pyr-His-Trp-Ser-Tyr-Gly-Leu-Arg-Pro-Gly). In some embodiments, the peptide comprising LHRH is a peptide comprising the amino acid sequence of SEQ ID NO: 1.

In some embodiments, the LHRH or its analog can be a LHRH agonist or a LHRH antagonist. Suitable LHRH agonists include nonapeptides and decapeptides represented by the formula: L-pyroglutamyl-L-histidyl-L-tryptophyl-L-seryl-L-tyrosyl-X-Y-arginyl-L-prolyl-Z (SEQ ID NO: 2), wherein X is D-tryptophyl, D-leucyl, D-alanyl, iminobenzyl-D-histidyl, 3-(2-naphthyl)-D-alanyl, O-tert-butyl-D-seryl, D-tyrosyl, D-lysyl, D-phenylalanyl, 1-benzyl-D-histidyl or N-methyl-D-alanyl and Y is L-leucyl, D-leucyl, N.sup..alpha.-methyl D-leucyl, N.sup..alpha.-methyl-L-leucyl or D-alanyl and wherein Z is (Aza)glycyl-NHR₁ or NHR₁ wherein R₁ is H, lower alkyl or lower haloalkyl. Lower alkyl includes straight—or branched-chain alkyls having 1 to 6 carbon atoms, e.g., methyl, ethyl, propyl, pentyl or hexyl, isobutyl, neopentyl and the like. Lower haloalkyl includes straight—and branched-chain alkyls of 1 to 6 carbon atoms having a halogen substituent, e.g., —CF3, —CH2CF3, —CF2CH3. Halogen means F, Cl, Br, I with Cl. In some embodiments, the LHRH analog is a nonapeptide wherein, Y is L-leucyl, X is an optically active D-form of tryptophan, serine (t-BuO), leucine, histidine (iminobenzyl), and alanine.

In some embodiments, the decapeptides include [D-Trp⁶]-LHRH wherein X=D-Trp, Y=L-leucyl, Z=glycyl-NH2, [D-Phe⁶]LHRH wherein X=D-phenylalanyl, Y=L-leucyl and Z-glycyl-NH2) or [D-Nal(2)⁶]LH-RH which is [(3-(2-naphthyl)-D-Ala⁶]LHRH wherein X=3-(2-naphthyl)-D-alanyl, Y=L-leucyl and Z=glycyl-NH2).

In some embodiments, the LHRH analog include alpha-aza analogues of the natural LHRH, especially, [D-Phe⁶, Azgly¹⁰]-LHRH, [D-Tyr(Me)⁶, Azgly¹⁰]-LHRH, and [D-Ser-(t-BuO)⁶, Azgly¹⁰]-LHRH, (see U.S. Pat. Nos. 4,100,274, 4,024,248 and 4,118,483 incoporated herein by reference in their entireties).

In some embodiments, the LHRH analogs include but are not limited to [D-Ala6]-LHRH; [DLys6]-LHRH; [D-Trp6]-LHRH; [Trp6]-LHRH; [D-Phe6]-LHRH; [D-Leu6]-LHRH; [D-Ser(t-Bu)61-LHRH; [D-His(Bzl)61]-LHRH; [D-Nal(2)6]1-LHRH;]Gln8]-LHRH; [His(3-Methyl)2]-LHRH; [des-Gly10, D-Ala6]-LHRH ethylamide; [-Me-Leu7]-LHRH; [des-Gly10, D-His2, D-Trp6, Pro9]-LHRH ethylamide; [des-Gly10, D-His(Bzl)6]-LHRH ethylamide; [des-Gly10, D-Phe6]-LHRH ethylamide; [aza-Gly110]-LHRH; [D-Ala6, N-Me-Leu7]-LHRH; [D-His(benzyl)6]-LHRH fragment 3-9 ethylamide; [D-His(Bzl)6]-LHRH fragment 1-7; [D-His(Bzl)6]-LHRH fragment 2-9; [D-His(Bzl)61]-LHRH fragment 4-9; [DHis(Bzl)6]-LHRH fragment 5-9; [D-pGlul, DPhe2,D-Trp3,6]-LHRH; [D-Ser4]-LHRH; [D-Trp6]-LHRH-Leu-Arg-Pro-Gly-NH2; [des-Gly10, D-Ala6]-LHRH ethylamide; [des-Gly110,12 D-His(Bzl)61]-LHRH ethylamide; [des-Gly10, D-His2, D-Trp6, Pro9]-LHRH ethylamide; [des-Gly10, D-Phe6]-LHRH ethylamide; [des-Gly10, D-Ser4, D-His(Bzl)6, Pro9]-LHRH ethylamide; [des-Gly10, D-Ser4, D-Trp6, Pro9]-LHRH ethylamide; [des-Gly10, D-Trp6, D-Leu7, Pro9]-LHRH ethylamide; [des-Gly10, D-Trp6]-LHRH ethylamide; [des-Gly10, D-Tyr5, D-Trp6, Pro9]-LHRH ethylamide; [des-pGlul]-LHRH; [His(3-Methyl)21]-LHRH; [Hyp9]-LHRH; Formyl-[D-Trp6]-LHRH Fragment 2-10; LHRH Fragment 1-2; LHRH Fragment 1-4; LHRH fragment 4-10; LHRH fragment 7-10 ihydrochloride; [D-Trp6]-LHRH fragment 1-6; nafarelin; deslorelin; a EHWSYGLRPG sequence; leuprolide; leuprolide acetate (Lupron™); Goserelin; Histrelin; Triptorelin; Buserelin; Cetrorelix; Ganirelix; Antide (Ala-Phe-Ala-Ser-Lys-Lys-Leu-Lys-Pro-Ala); Abarelix; Teverelix; Degarelix; Nal-Glu (D-2-Nal-p-Chloro-D-Phe-BETA-(3-Pyridyl)-D-Ala-Ser-Arg-D-Glu-Leu-Arg-Pro—D-Ala); or Elagolix (NBI-56418).

In some embodiments, the LHRH or LHRH analog comprises a sodium or acetate salt. In some embodiments, the LHRH analog is [DLys⁶] LHRH (pyroGlu-His-Trp-Ser-Tyr-DLys-Leu-Arg-Pro-Gy-NH2, Seq ID NO: 3). In some embodiments, the LHRH analog comprises the amino acid sequence of SEQ ID NO: 3. In some embodiments, the glutamic acid residue is pyroglutamic acid. In some embodiments, the amino acid sequence of the LHRH analog consists of SEQ ID NO: 3.

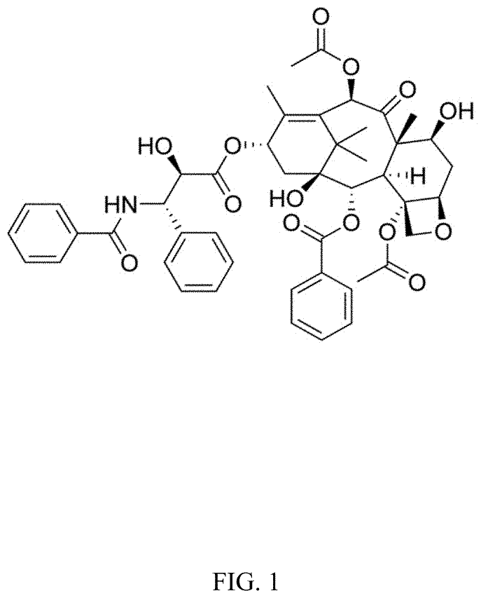

In some embodiments, the drug conjugate to LHRH or its analog can be an active agent comprising paclitaxel or paclitaxel active agent (PTX, FIG. 1). In some embodiments, the LHRH-conjugated paclitaxel cancer drugs are synthesized by conjugating [D-Lys6]LHRH to paclitaxel at the epsilon (ε) amino side chain of the D-Lys6 moiety. In some embodiments, the −Trp residue is implicated in the binding to the breast cancer LHRH receptor. In some embodiments, the conjugate can be formed by conjugating [D-Lys6]LHRH to paclitaxel at the epsilon (F) amino side chain of the D-Lys6 moiety at position 6 of the [D-Lys⁶]LH-RH (pyroGlu-His-Trp-Ser-Tyr-d-Lys-Leu-Arg-Pro-Gly-NH2). The conjugation can be successfully accomplished without the loss of the drugs' abilities to bind to LHRH receptors

In the case of PTX, the native lysine ε-amines groups of the LHRH-peptide were targeted for the drug coupling as shown below:

$$OH \text{-} {2}^{′} \text{-} PTX + Succinic Anhydride \to PTX \text{-} {2}^{′} \text{-} {O}_{2} PTX {O}_{2} {OCCH}_{2} {CH}_{2} {CO}_{2} H ( PTXSCT )$$

$$LHRN \text{-} {NH}_{2} + PTXSCT \to \frac{NHS / EEDG}{DMF} \to LHRH \text{-} NH \text{-} PTX ( LHRH \text{-} PTX )$$

The targeting moieties were attached to PTX via the 2-hydroxyl group (on one of its side chains) in the presence of the heterobifunctional linkers. The major function of these linkers is to hold the segment of the drug and the LHRH peptide together sufficiently enough for the ligands to be attached specifically to the target receptors on the cancer cells/tumors [Safavy, A et al. (2003). Bioconjugate chemistry, 14 2, 302-10].

In some embodiments, the PTX is conjugated to LHRH by esters linkage. In some embodiments, a linker can be used to conjugate the LHRH or its analog to the drug of interest, for example, by covalent bonding. In some embodiments, a linker having a hydrophilic portion or a hydrophilic linker can be used to conjugate the drug to the LHRH or LHRH analog. Various branched or linear hydrophilic linkers can be used, in which the hydrophilic portion can form the backbone of the linker or be pendant to or attached to the backbone of the linker. In some embodiments, the LHRH or its analog can be cross-linked to the drug of interest. In some embodiments, the hydrophilic linker can be a linker that activates carboxyl groups for spontaneous reaction with primary amines. In some embodiments, the hydrophilic linker can be N-hydroxysuccinimide (NHS). In some embodiments, the hydrophilic linker can be Sulfo-NHS. In some embodiments, the linker can be a water-soluble carbodiimide crosslinker. In some embodiments, the linker can be 1-ethyl-3-(3-dimethylaminopropyl)carbodiimide hydrochloride (EDC). EDC is water-soluble carbodiimide crosslinker that activates carboxyl groups for spontaneous reaction with primary amines.

The presence of the hydrophilic linker (NHS) creates sites for the reaction with the methoxy group (—OCH3) that is present in the PTX molecule. The methoxy group (—OCH3) has high electron density and has a tendency to attack the nucleophilic center of the carbonyl group that is present in the NHS. With the presence of EDC, the high electron density attacks the PTX linkages, causing the electrostatic cleavage of the proton from the N—H group, thus linking the LHRH or LHRH analog. The reaction with the secondary amine (NH) creates stable amide linkages that do not easily break down. Thus, in the presence of the LHRH molecules, NHS ester crosslinks or couples to the α-amines to the lysine side chains and to the α-amines in the N-terminals.

In some embodiments, the conjugation can take place in the presence of EDC/NHS crosslinker. EDC is a carboxyl and amine-reactive zero-length crosslinker. The EDC/NHS is heterogeneous crosslinking process that is facilitated by covalent binding strategy of the amino or carboxyl groups on peptide to the free carboxyl or amino groups on drug/activated drug. In some embodiments, the drug that can be conjugated with EDC/NHS linker has a carboxyl and/or an amino group or can be activated such that the drug possesses a carboxyl and/or an amino group.

In some embodiments, the structures produced by the conjugation reactions are characterized using Fourier Transform Infra-Red (FTIR) and Nuclear Magnetic Resonance (NMR) spectroscopy.

### Compositions Comprising LHRH-Conjugated Paclitaxel

Other aspects of the disclosure relate to the compositions comprising an effective amount of LHRH-conjugated paclitaxel.

As used herein “pharmaceutical formulation”, “pharmaceutical composition”, “formulation”, or “composition” are used interchangeably. In some embodiments, pharmaceutical composition comprises the LHRH-conjugated paclitaxel and a physiologically acceptable carrier.

Pharmaceutical compositions include solid formulations, liquid formulations, e.g. aqueous, solutions that may be directly administered, and lyophilized powders which may be reconstituted into solutions by adding a solution (e.g. diluent) before administration.

In some embodiments, the composition can be formulated for oral, parental, intravenous, intranasal, intratumoral, and intramuscular administration.

In some embodiments, the pharmaceutical compositions provided herein can be administered parenterally (e.g., by intravenous, intramuscular, or subcutaneous injection). In some embodiments, the pharmaceutical compositions provided herein can be administered orally. In some embodiments, the pharmaceutical compositions provided herein can be administered intranasally. In some embodiments, the pharmaceutical compositions provided herein can be administered rectally. In some embodiments, the pharmaceutical compositions provided herein can be administered intratumorally. As used herein, the terms “physiologically acceptable” and “pharmaceutically acceptable” are used interchangeably and mean approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the active agent is administered. Physiologically acceptable carriers can be sterile liquids, such as water and oils, including those of petroleum, animal, vegetable or synthetic origin (e.g., peanut oil, soybean oil, mineral oil, or sesame oil). Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water and ethanol. The composition, if desired, can also contain minor amounts of wetting or emulsifying agents, or pH buffering agents.

For example, the pharmaceutical compositions according to some embodiments can comprise one or more excipients, one or more buffers, one or more diluents, one or more additives or combinations thereof that are formulated for administration to a subject in need thereof. Pharmaceutically-acceptable excipients and carrier solutions are well-known to those of ordinary skill in the art. Pharmaceutically acceptable auxiliary substances may also be included to approximate physiological conditions, such as pH adjusting and buffering agents, tonicity adjusting agents, dispersing agents, suspending agents, wetting agents, detergents, antioxidants, stabilizers, chelating agents, disintegrants, binders, and preservatives. For example, the pharmaceutical compositions can comprise one or more detergents/surfactants (e.g. PEG, Tween (20, 80, etc.), Pluronic), excipients, antioxidants (e.g. ascorbic acid, methionine), coloring agents, flavoring agents, preservatives, stabilizers, buffering agents, chelating agents (e.g. EDTA), suspending agents, isotonizing agents, binders, disintegrants, lubricants, and fluidity promoters.

Pharmaceutical compositions may be formulated for any appropriate manner of administration, including, for example, parenteral, intranasal, topical, oral, rectal, or local administration. The pharmaceutical compositions may be formulated according to conventional pharmaceutical practice.

These compositions can be formulated in a form that suits the mode of administration, such as solutions, suspensions, emulsions, tablets, pills, capsules, powders, aerosols and sustained-release formulations.

Oral formulation can include standard carriers such as pharmaceutical grades of mannitol, lactose, starch, magnesium stearate, sodium saccharine, cellulose, magnesium carbonate, etc. Examples of suitable pharmaceutical modes of administration and carriers are described in “Remington: The Science and Practice of Pharmacy,” A.R. Gennaro, ed. Lippincott Williams & Wilkins, Philadelphia, Pa. (21.sup.st ed., 2005).

Oral dosage forms may be tablets, troches, lozenges, aqueous or oily suspensions, dispersible powders or granules, emulsion, hard or soft capsules, or syrups or elixirs. Such compositions may further comprise one or more components such as sweetening agents flavoring agents, coloring agents and preserving agents. Tablets can contain the active agent in admixture with physiologically acceptable excipients that are suitable for the manufacture of tablets. Such excipients include, for example, inert diluents, granulating and disintegrating agents, binding agents and lubricating agents. Oral dosage forms can be hard gelatin capsules wherein the active agent is mixed with an inert solid diluent, or as soft gelatin capsules wherein the active agent is mixed with water or an oil medium. Aqueous suspensions can comprise the active agent in admixture with one or more excipients suitable for the manufacture of aqueous suspensions. Such excipients include suspending agents and dispersing or wetting agents. The active agent can be formulated as a dispersible powder and granule suitable for preparation of an aqueous suspension by the addition of water, a dispersing or wetting agent, suspending agent and one or more preservatives.

The composition can be formulated as a suppository, with traditional binders and carriers such as triglycerides.

In some embodiments, the pharmaceutical compositions provided herein are administered parenterally. In some embodiment, the pharmaceutical compositions are administered to a subject in need thereof systemically, e.g., by IV infusion or injection. For parenteral administration, the LIRH-conjugated PTX can either be suspended or dissolved in the carrier. Among the acceptable carriers that may be employed are water, buffered water, Ringer's solution, saline or phosphate-buffered saline, U.S.P., and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectable compositions. In some embodiments, the pharmaceutical composition is sterile injectable composition. In some embodiments, the sterile injectable composition is a sterile injectable solution or suspension in a non-toxic parenterally acceptable diluent or solvent.

In certain embodiments, a “therapeutically effective amount” of disclosed conjugated drug or microspheres comprising the conjugated drug is that amount effective for treating, alleviating, ameliorating, relieving, delaying onset of, inhibiting progression of, reducing severity of, and/or reducing incidence of one or more symptoms or features of cancer, for example, TNBC.

In some embodiments, the conjugated drug may be administered to a subject in such amounts and for such time as is necessary to achieve the desired result (i.e., treatment of cancer). In some embodiments, microspheres may be administered to a subject in such amounts and for such time as is necessary to achieve the desired result (i.e., remission of cancer). In certain embodiments, a “therapeutically effective amount” is that amount effective for treating, alleviating, ameliorating, relieving, delaying onset of, inhibiting progression of, reducing severity of, and/or reducing incidence of one or more symptoms or features of cancer, for example TNBC.

In some embodiments, the effective amount can depend on the patient, the extent of the cancer, age, gender, weight, etc. Such effective amounts can be readily determined by an appropriately skilled practitioner, taking into account the severity of the condition to be treated, the route of administration, and other relevant factors—well known to those skilled in the art. Such a person will be readily able to determine a suitable dose, mode and frequency of administration.

As used herein, the term “inhibits growth of cancer cells” or “decreases growth of cancer cells” refers to any slowing of the rate of cancer cell proliferation and/or migration, arrest of cancer cell proliferation and/or migration, or killing of cancer cells, such that the rate of cancer cell growth is reduced in comparison with the observed or predicted rate of growth of an untreated control cancer cell. The term “inhibit”, “decease” or “inhibition” refers to a reduction in size or disappearance of a cancer cell or tumor, as well as to a reduction in its metastatic potential. In some embodiment, such decrease or inhibition may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by direct or indirect measurement of cancer cell or tumor size. In human cancer patients, such measurements generally are made using well known imaging methods such as magnetic resonance imaging, computerized axial tomography and X-rays.

Compositions described herein can be administered to provide the intended effect as a single or multiple dosages, for example, in an effective or sufficient amount. In some embodiments, the conjugated drug can be administered at a dose corresponding from about 1 mg/kg to about 1 g/kg, about 1 mg/kg to about 100 mg/kg, about 1 mg/kg to about 10 mg/kg, about 1 mg/kg to about 100 mg/kg.

In some embodiments, a pharmaceutical composition or formulation includes the combination of the conjugated drug and one or more active agent. In some embodiments, the active agent is an anti-cancer active agent. In some embodiments, the anti-cancer active agent comprises an alkylating agent, anti-metabolite, plant extract, plant alkaloid, nitrosourea, hormone, nucleoside analog or a nucleotide analog. In some embodiments, the anti-cancer active agent comprises gemcitabine, 5-fluorouracil, cyclophosphamide, azathioprine, cyclosporin A, prednisolone, melphalan, chlorambucil, mechlorethamine, busulphan, methotrexate, 6-mercaptopurine, thioguanine, cytosine arabinoside, AZT, 5-azacytidine (5-AZC), bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, taxol (paclitaxel), vinblastine, vincristine, doxorubicin, dibromomannitol, irinotecan, topotecan, etoposide, teniposide, or pemetrexed.

In some embodiments, the compositions of the present disclosure can further comprise microspheres, microparticles, nanospheres and the like. In some embodiments, the compositions can be formulated for administration to one or more cells, tissues, organs, or body of a human undergoing treatment for cancer, for example, TNBC.

According to some aspects of the disclosure, polymeric microspheres or particles loaded with LHRH-PTX compositions are provided. Biocompatible polymers may be used and may be, in some embodiments, selected from the group consisting of diblock poly(lactic) acid-poly(ethylene)glycol copolymer, poly(lactic) acid, diblock poly(lactic-co-glycolic) acid-poly(ethylene)glycol copolymer, poly(lactic-co-glycolic) acid, and mixtures thereof.

In some embodiments, biocompatible polymeric materials such as poly-lactide-co-glycolide (PLGA) and polyethylene glycol (PEG) can be used for controlled localized and targeted cancer drug delivery. Poly (ethylene glycol) (PEG) is a hydrophilic polymer that decreases its interactions with blood components. The proportion of poly lactic acid (PLA) and poly glycolic acid (PGA) in poly lactic acid co glycolic acid (PLGA) can be used to control the degradation rates or drug release rates during controlled release from PLGA. In some embodiments, the microsphere can have an optimized ratio of the biocompatible polymers such that an effective amount of conjugated drug is associated with the microsphere for treatment of TNBC. In some embodiments, the blend consists of PLGA and PEG polymer in the ratio of 1:1, but other proportion may be used depending on desired release rate. In some embodiments, the poly(ethylene)glycol (PEG) has a number average molecular weight of about 4 to about 10 kDa. In some embodiments, the poly(ethylene)glycol (PEG) has a number average molecular weight of 8 kDa.

In general, the “microspheres” refers to any particle having a mean size of less than 1500 nm, e.g., about 80 nm to about 1300 nm. Disclosed microspheres may include nanoparticles having a diameter of about 80 to about 1300 nm, about 90 to about 1300 nm, about 100 to about 1300 nm, about 200 to about 1300 nm, about 300 to about 1300 nm, about 400 to about 1300 nm, about 500 to about 1300 nm, about 600 to about 1300 nm, about 700 to about 1300 nm, about 800 to about 1300 nm, about 900 to about 1300 nm, about 1000 to about 1300 nm, about 1100 to about 1300 nm, about 1200 to about 1300 nm, about 80 to about 1000 nm, about 90 to about 1000 nm, about 100 to about 1000 nm, about 200 to about 1000 nm, about 300 to about 1000 nm, about 400 to about 1000 nm, about 500 to about 1000 nm, about 600 to about 1000 nm, about 700 to about 1000 nm, about 800 to about 1000 nm, about 900 to about 1000 nm, about 80 to about 500 nm, about 90 to about 500 nm, about 100 to about 500 nm, about 200 to about 500 nm, about 300 to about 500 nm, about 400 to about 500 nm, or any value therebetween.

In some embodiments, the mean particle sizes of the microsphere is between 0.84 and 1.23 μm.

In some embodiments, blend of polymers (PLGA and PEG) can be used to encapsulate targeted drugs (LHRH-PTX) for the enhancement of sustained and localized delivery of targeted drugs for breast cancer treatment, in particular TNBC. In some embodiments, the encapsulated form LHRH-PTX formulation can be used to target LHRH-PTX to the target cells/tissue for a controlled and prolong release period. In some embodiments, the encapsulated form LHRH-PTX formulation can provide an extended release of the drug over periods of several days to several months. For example, the encapsulated form LHRH-PTX formulation can provide an extended release of the drug over periods of one week, two weeks, three weeks, four weeks, five weeks, six weeks, seven weeks, eight weeks, nine weeks, or more. In some embodiments, the encapsulated form LHRH-PTX formulation can provide an extended release of the drug over periods of 62 days.

In some embodiments, administration of the encapsulated form LHRH-PTX formulation results in a decrease the viability of TNBC cells. For example, decrease can include but is not limited to a 5%, 10%, 20%, 30%, 40%, 50%, 60%, 70%, 80%, 90% or 100% (or any percentage of reduction in between) decrease of the viability of TNBC cells.

Microspheres disclosed herein may be combined with pharmaceutically acceptable carriers to form a pharmaceutical composition. The carriers may be chosen based on the route of administration as described below, the location of the target tissue, the drug being delivered, the time course of delivery of the drug, etc.

### Kits

Aspects of the disclosure provide kits including the conjugated drug, and pharmaceutical formulations thereof, packaged into suitable packaging material. A kit optionally includes a label or packaging insert including a description of the components or instructions for use in vitro, in vivo, or ex vivo, of the components therein. The term “packaging material” refers to a physical structure housing the components of the kit. The packaging material can maintain the components sterilely, and can be made of material commonly used for such purposes (e.g., ampules, vials, tubes, etc.). Each component of the kit can be enclosed within an individual container and all of the various containers can be within a single package. In some embodiments, the kits can be designed for sterile, stable and/or cold storage. The cells in the kit can be maintained under appropriate storage conditions until used. Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

### Methods of Treatment

In some embodiments, the present disclosure provides methods of treatment of tumor, cancer or malignancy where the cells express one or more receptors that bind to LHRH or an LHRH analog. In some embodiments, the conjugates of the present disclosure may be used for the treatment of solid cancerous tumors. For example, the conjugates of the present disclosure may be used to treat breast, pancreatic, uterine and ovarian, testicular, gastric or color, hepatomas, adrenal, renal and bladder, lung, head and neck cancers and tumors.

In some embodiments, the methods comprise administering the pharmaceutical composition to a subject having tumor, cancer or malignancy including but not limited to ovarian cancers, endometrial cancers, carcinoma, sarcoma, lymphoma, leukemia, adenoma, adenocarcinoma, melanoma, glioma, glioblastoma, meningioma, neuroblastoma, retinoblastoma, astrocytoma, oligodendrocytoma, mesothelioma, or reticuloendothelial neoplasia. In some embodiments, sarcoma comprises a lymphosarcoma, liposarcoma, osteosarcoma, chondrosarcoma, leiomyosarcoma, rhabdomyosarcoma or fibrosarcoma.

In some embodiments, the conjugates of the present disclosure are administered to treat a triple negative breast cancer (TNBC). It has been shown that LHRH receptors are expressed on TNBC tissues (Engel J, et al., Expert Opin Investig Drugs. 2012, 21: 891-899). Furthermore, common and conventional breast cancer diagnosis techniques target ER, PR and HER2 receptors. Thus, in the case TNBC, it is often difficult to detect and treat with conventional targeted hormonal therapy. This results in their relatively poor prognosis, aggravated side effects, aggressive tumor growth and limited targeted therapies. Other therapeutic approaches, such as chemotherapy and radiation therapy, lack the specificity that is needed for the effective treatment of TNBC. They also result in severe side effects.

Different breast cancer cells have been shown to have exhibit or acquire intrinsic resistance to chemotherapy (Kydd et al., Pharmaceutics, 2017, 9, 46). Such drug resistance is often associated with complicated tumor microenvironments. Furthermore, in case of bulk chemotherapy, only a very small fraction of the drug may reach the tumor sites of interest. This results in several side effects that are associated with drug interactions with non-tumor/healthy tissue and organs. In most cases, targeted cancer drug delivery systems have been developed for the treatment of tumors that over-expressed receptors that can attach specifically to antibodies, peptides and hormonal receptors. Cancer drugs have also been developed to bind specifically to HER2 receptors, progesterone and estrogen receptors. However, TNBC presents challenges since it is not well targeted by conventional cancer drugs. There is, therefore a need to develop targeted chemotherapeutic drugs that are more effective in the targeting and treatment of TNBC.

As used herein a “subject” or a “patient” refers to any animal. In some embodiments, the animal is a mammal. In some embodiments, the subject is a human. Any animal can be treated using the methods and composition of the present disclosure.

The pharmaceutical composition can be administered in single or multiple doses, optionally in combination with one or more other compositions therapeutic agents for any duration of time (e.g., for hours, days, months, years) (e.g., 2, 4, 5, 6, 7, 8, 9, 10, 11, or 12 times per hour, day, week, month, or year). In some embodiments, a single dose per day comprising the drug can be administered to the subject in need thereof to treat TNBC.

In some embodiments, the pharmaceutical composition can be administered to a mammal (e.g., a human) continuously for 1, 2, 3, or 4 hours; 1, 2, 3, or 4 times a day; every other day or every third, fourth, fifth, or sixth day; 1, 2, 3, 4, 5, 6, 7, 8, 9, or 10 times a week; biweekly; 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 times a month; bimonthly; 1, 2, 3, 4, 5, 6, 7, 8, 9, or 10 times every six months; 1, 2, 3, 4, 5, 6, 7, 8, 9, 10, 11, 12, 13, 14, 15, 16, 17, 18, 19, or 20 times a year; or biannually. It will be apparent that a pharmaceutical composition may, but need not, be administered at different frequencies during a therapeutic regimen.

As used herein the term “treating” comprises administering the drug of the present disclosure to measurably reduce (e.g., for about 1-5%, 5-10%, 10%-20%, about 20%-40%, about 50%, about 40%-60%, about 60%-80%, about 80%-90%, 90-95%) shrink or eliminate tumors at early, mid and late stages of triple negative breast cancer. Treatment can therefore result in inhibiting, reducing or preventing a disorder, disease or condition, or an associated symptom or consequence, or underlying cause; inhibiting, reducing or preventing a progression or worsening of a disorder, disease, condition, symptom or consequence, or underlying cause; or further deterioration or occurrence of one or more additional symptoms of the disorder, disease condition, or symptom.

In some embodiments, the method of treatment results in partial or complete destruction of the cell mass, volume, size etc. of the tumor. As used herein, “reduction”, “decrease” or “reduce” refer to any change that results in a smaller amount of a symptom, condition, disease or tumor size. For example, a reduction or decrease can be a change in TNBC such that the symptoms or tumor size are less than previously observed. Thus, for example, a reduction or decrease can include but is not limited to a 10%, 20%, 30%, 40%, 50%, 60%, 70%, 80%, 90% or 100% (or any percentage of reduction in between) decrease in the symptoms associated with TNBC or tumor size.

As used herein, the term “therapeutically effective amount” means a dose that is sufficient to achieve a desired therapeutic effect for which it is administered.

In some embodiments, the methods further comprise administering a therapeutically effective amount of the conjugated drug and one or more active agent. In some embodiments, the administration is concurrent. In some embodiments, the administration is sequential.

In some embodiments, the active agent is an anti-cancer active agent. In some embodiments, the anti-cancer active agent comprises an alkylating agent, anti-metabolite, plant extract, plant alkaloid, nitrosourea, hormone, nucleoside analog or a nucleotide analog. In some embodiments, the anti-cancer active agent comprises gemcitabine, 5-fluorouracil, cyclophosphamide, azathioprine, cyclosporin A, prednisolone, melphalan, chlorambucil, mechlorethamine, busulphan, methotrexate, 6-mercaptopurine, thioguanine, cytosine arabinoside, AZT, 5-azacytidine (5-AZC), bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, taxol (paclitaxel), vinblastine, vincristine, doxorubicin, dibromomannitol, irinotecan, topotecan, etoposide, teniposide, or pemetrexed.

In some embodiments, administration of the compositions described herein result in shrinkage or elimination of tumors at early, mid and late stages of breast progression. For example, the effects of the LHRH-conjugated paclitaxel drug were then compared in in vitro experiments using TNBC cell line (MDA MB 231 cell) and in vivo experiments on an athymic nude mouse model injected with TNBC to induce xenograft tumor. The conjugated LHRH-paclitaxel was shown to shrink or eliminate tumors at early, mid and late stages of breast progression.

The in vivo studies show that the injection of 10 mg/kg of LHRH-conjugated paclitaxel results in the elimination of early stage breast tumors. In the case of mid stage tumors (formed 21-days after tumor induction) and late stage tumors (formed 28-days after tumor induction), significant shrinkages in the tumor sizes (91% after 21 days) and (90.2% after 28 days) were observed for LHRH-conjugated paclitaxel.

In some embodiments, the LHRH-conjugated drugs have adhesion forces/interactions between the LHRH-conjugated drugs (e.g. PTX) and breast cancer tissue that is at least 3 times, at least 4 times, or more, higher than between unconjugated drugs (e.g. PTX) and breast tumor. For example, the adhesion forces/interactions between the LHRH-conjugated drugs (e.g. PTX) and breast cancer tissue were shown to be three times those between unconjugated drugs (e.g. PTX) and early/mid stage breast tumor, but four times in those of late stage breast cancer tumors.

In some embodiments, administration of the conjugated drug enhances the specific targeting of the drug. Furthermore, ex vivo histopathological tests revealed no evidence of physiological changes due to LHRH-conjugated drug administration. No clinical signs, differences in mortality, or changes in body weight, were observed in the mice after treatment with LHRH-PTX. Hence, the current results show that LHRH-conjugated PTX enhances the specific targeting of TNBCs.

In some embodiments, the conjugated drugs can be formulated for intravenous administration at a dose between about 100 mg/m2 to about 250 mg/m2, about 100 mg/m2 to about 200 mg/m2, about 100 mg/m2 to about 175 mg/m2, about 100 mg/m2 to about 150 mg/m2, about 150 mg/m2 to about 250 mg/m2, about 150 mg/m2 to about 200 mg/m2, about 150 mg/m2 to about 175 mg/m2, about 175 mg/m2 to about 250 mg/m2, about 175 mg/m2 to about 200 mg/m2, about 200 mg/m2 to about 250 mg/m2, for example 175 mg/m2. In some embodiments, the conjugated drugs can be formulated for an intratumoral administration.

In some embodiments, the formulation can be administered intravenously or intratumorally every 1 to 4 weeks for 2-8 cycles. In some embodiments, the formulation can be administered intravenously or intratumorally every 3 weeks for 4 treatment cycles. In some embodiments, the formulation can be administered intravenously or intratumorally every week, every two weeks, every three week, every four weeks for up to 30 weeks. In some embodiments, the intravenous administration can be used in combination with the conjugated drug loaded microspheres. In some embodiments, the intravenous administration can be used in combination with intratumoral administration of the conjugated drug loaded microspheres. For example, an initial dosage of the conjugated drugs can be administered intravenously and the conjugated drug loaded microspheres can be administered in subsequent dosages for a period of one or more treatment cycles. In some embodiments, the microspheres can be formulated to deliver the therapeutic load over a period of about 1 to 8 weeks, in some embodiments, over a period of 6 weeks. In some embodiments, the microspheres can be delivered into the tumor or into tissue in proximity to the tumor or from which the tumor has been excised.

The following examples are put forth so as to provide those of ordinary skill in the art with a complete disclosure and description of how to make and use the compositions and methods of the disclosure, and are not intended to limit the scope of what the inventors regard as their disclosure.

### EXAMPLES

### Example 1: LHRH-Paclitaxel Conjugates

### Paclitaxel Conjugation

Paclitaxel, (N-hydroxysuccinimide (NHS), 1-ethyl-3-(3-dimethylaminopropyl)carbodiimide hydrochloride (EDC HCl), Alamar Blue Assay (ABA) kits and Dubecco Phospate Buffer (DPBS) were purchased from Thermofisher Scientific (Waltham, Mass., USA). N,N-Dimethylformamide (DMF), 2-Ethoxy-1-ethoxycarbonyl-1,2-dihydroquinoline (EEDQ), Dimethyl sulfoxide (DMSO), [D-Lys6]LHRH peptide and silica were all obtained from Sigma-Aldrich Co. LLC, (St. Louis, Mo. USA). Also, 3 kDa Amicon Ultra-4 Centrifugal Filters Units and an Amicon Pro Purification System were purchased from Millipore Sigma (Burlington, Mass., USA).

The paclitaxel (PTX) #P3456 that was used in the study was purchased from Thermofisher Scientific (Waltham, Mass., USA). It was activated with 2-hydroxyl groups. Since the coupling of PTX directly to [D-Lys6]LHRH peptides was not favorable, a two-step coupling process was used to couple LHRH to PTX. First, esters of PTX were formed by modifying a method reported by Deutsch et al. [30] to form 2′-O-paclitaxel succinate (a hemisuccinate). This was done using PTX purchased from Thermofisher Scientific (Waltham, Mass., USA) and succinic anhydride. These were dried for 24 h in the presence of silica gel that was fused with calcium chloride at room temperature (˜23° C.) in a high-vacuum desiccator.

The dried PTX was then dissolved in anhydrous pyridine followed by the addition of a solid form of succinic anhydride. The combined solution was then kept at room-temperature (˜23° C.) under argon gas in a 3-neck sealed flask. This was done for 12 h to form 2′-O-paclitaxel succinate (PTXSCT). Silica gel was then used to purify the resulting solution via column chromatography with chloroform as a solvent (for column packing and product loading).

The conjugation of PTXSCT to [D-Lys6]LHRH was done by initially activating PTXSCT with freshly prepared NHS and EEDQ linker in dry DMF and gently stirred at 4° C. for 3 h. The resulting solution containing DMF solution of the PTXSCT activated ester was then added to the [D-Lys6]LHRH and gently vortexed at 600 rpm for 6 hours at 4° C. to form LHRH-conjugated paclitaxel drug. The conjugated drug molecule was purified using a combination of 3 kDa Amicon Ultra-4 Centrifugal Filters Units, and a Amicon Pro Purification System. The conjugation was confirmed with FTIR, and further characterized with LC-MS.

In the case of PTX, the native lysine-amines groups of the [D-Lys6]LHRH-peptide were targeted for the drug coupling (See Equations 4 and 5).

$$\begin{matrix}OH - {2}^{′} - PTX + Succinic Anhydride \to PTX - {2}^{′} - {O}_{2} PTX {O}_{2} {OCCH}_{2} {CH}_{2} {CO}_{2} H \\ ( PTXSCT ) & ( 4 ) \\ LHRN - {NH}_{2} + PTXSCT \to \frac{NHS / EEDG}{DMF} \to LHRN - NH - PTX \\ ( PTXLHRH ) & ( 5 )\end{matrix}$$

The targeting moieties were attached to PTX via the 2-hydroxyl group (on one of its side chains) in the presence of the heterobifunctional linkers. The major function of these linkers is to hold the segment of the drug and the LHRH peptide together sufficiently enough for the ligands to be attached specifically to the target receptors on the cancer cells/tumors.

### Drug Characterization (FTIR, NMR, LCMS)

PTX and its conjugated components, LHRH-PTX (as described in Example 1), were analyzed using Attenuated Total Reflectance Fourier Transform Infrared spectroscopy (ATR-FTIR) (IRSpirit, Shimadzu, Kyoto, Japan). The FTIR was set to the transition mode in an effort to investigate the functional groups, bonding types, and the nature of compounds that were formed.

A Bruker High-performance digital NMR Spectrometer AVANCE III TN 500 MHz was used to obtain 1HNMR in S(ppm). Drug samples were dissolved with deuterated chloroform (CDCl3) that was purchased from Cambridge Isotope (Tewksbury, Mass., USA) as solvents in 5 mm tubes (ChemGlass Life Science, Vineland, N.J., USA).

An Agilent 1200 LC/MS system with 6130 series (Santa Clara, Calif., USA) single-quadrupole was used to analyze the purity of the conjugated drugs. The drug samples were ionized using an electrospray source with polarity switching (±ESI). The Ionized species were analyzed at an m/z range between 180 and 1200. This was done using the gradient method under acidic conditions.

The mobile phase components were A1: 95% H₂O 5% acetonitrile containing 0.1% formic acid, B1: 5% H₂O 95% acetonitrile containing 0.1% formic acid. These were identified with a diode array detector that simultaneously monitors the following three UV wavelengths: 210 nm, 254 nm, and 277 nm. In each LC-MS test, 2 μl of sample was injected. Mobile Phase Composition: 5% B for 0.5 min., 8 min. gradient to 100% B, hold 1 min., 0.5 min. gradient to 5% B, hold 4 min. The total data acquisition time was also about 18 minutes per sample.

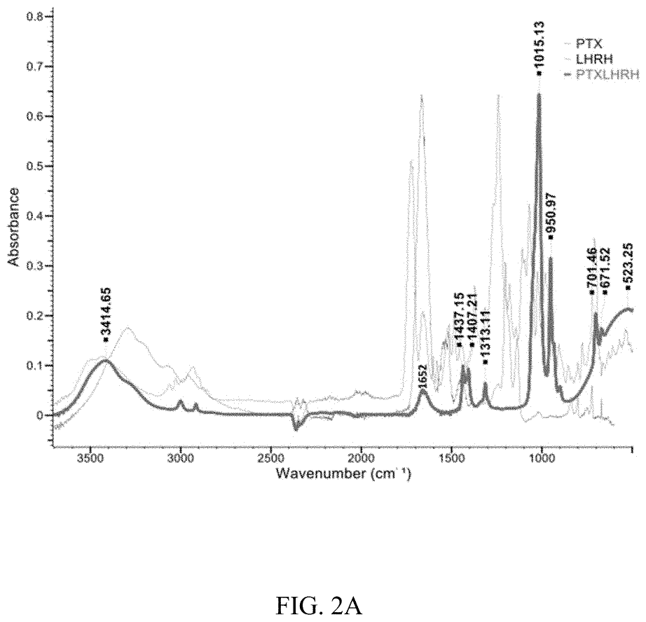

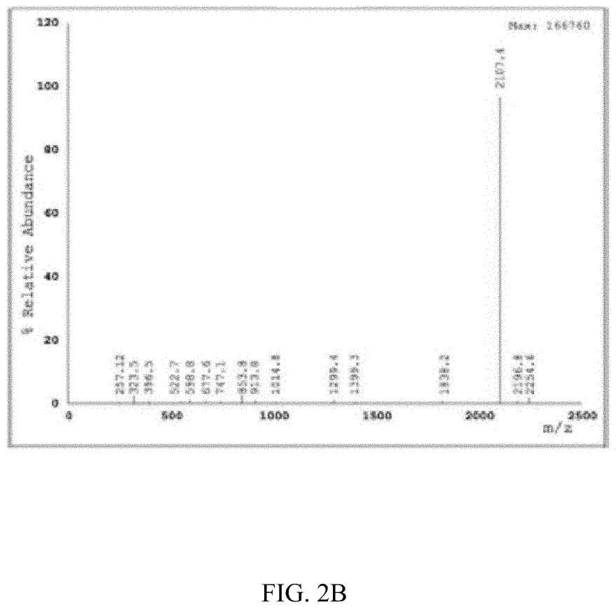

In reference to FIG. 2A, The FTIR spectral analysis of LHRH peptide revealed the presence of characteristic amine bands of —NH (˜1545 cm-1), which disappear after conjugation to PTX. The spectra shows the formation of the amide bond. The LHRH-conjugated drugs exhibited typical amide (covalent or peptide) bond signatures at around 1641 cm⁻¹.

In reference to FIG. 2B, the LC-MS spectra exhibited a molecular ion (m/z) peak of pigment that corresponds to the mass-to-charge ratio of LHRH-PTX with its molecular weights. In general, the LC-MS results are evidence that LHRH-conjugated PTX was formed during the conjugation process.

### EXPERIMENTAL PROCEDURE

### Cytotoxicity and Cancer Cell Viability Studies

The human triple negative cancer cell line (MDA MB 231) that was used to induced subcutaneous tumor, growth media (L15), and fetal bovine serum (FBS) were all purchased from American Type Culture Collection (ATCC, Manassas, Va., USA), while penicillin/streptomycin a cell medium supplement was obtained from Thermo Fisher Scientific, Inc. (Waltham, Mass., USA).

Athymic Nude-Foxn1nu strain mice with individual weights of ˜17 g were purchased from Envigo (South Easton, Mass., USA). All of the animals were approved for use in in animal experiments at the University of Massachusetts Medical School (Institutional Animal Care and Use Committee IACUC with docket #A2630-17).

The alamar blue cell viability and cytotoxicity assay was used to study the MDA MB 231 cells lines in the log phase of growth. MDA MB 231 cells were harvested with trypsin-EDTA in the presence of Dulbecco Phosphate Buffer (DPBS). 10⁴ cells/well were then seeded in 12-well plates in L15+ medium (L15 medium with cell medium supplement of FBS and penicillin/streptomycin). After a 3-hour attachment period (of the cells), respective concentrations of 15 μM, 25 μM and 30 μM of paclitaxel, LRH-conjugated paclitaxel (of Example 1) and DMSO (in culture medium) were added to the 12-well plates consisting of 10⁴ cells. Cell viability was monitored using the alamar blue cell viability and cytotoxicity reagent (Thermo Fisher Scientific, Waltham, Mass., USA) at times of 0 h, 18 h, 24 h, 48 h and 72 h, following drug addition. At each time point, the culture medium was replaced with 1 ml of 10% alamar blue solution (in culture medium). The 12-well plates were then incubated at 37° C. for different durations. After each time point, 100 l of the solution incubated with alamar blue solution (ABS) was transferred into triplicate wells of a black opaque 96-well plate (Thermo Fisher Scientific, Waltham, Mass., USA).

The fluorescence intensities of the cell medium supernatant incubated with ABS were measured at 544 nm excitation and 590 nm emission using a 1420 Victor3 multi-label plate reader (Perkin Elmer, Waltham, Mass., USA). The percentage of alamar blue reduction, the percentage difference in cell population between the treated and untreated cells, and the percentage of cell growth inhibition, were determined using a combination of the ABS and cell viability studies. In this way, the cytotoxicity of the respective conjugated drug molecules was obtained from equations 1 and 2 below. These gives:

$$\% Reduction = \frac{{FI}_{sample} - {FI}_{10 \% AB}}{{FI}_{100 \% R} - {FI}_{10 \% AB}} \times 100 \qquad ( 1 )$$

where, FIₛₐₘₚₗₑ=fluorescence intensity of the (treated or untreated) cells, FI_{10% AB}=fluorescence intensity of 10% alamar blue reagent (negative control) and FI_{10% R}=fluorescence intensity of 100% reduced alamar blue (positive control).

### Also,

$$\% Growth Inhibition = ( 1 - \frac{{FI}_{treated}}{{FI}_{untreated}} ) \times 100 \qquad ( 2 )$$

FI_{treated}=fluorescence intensity of treated cells and FI_{cells}=fluorescence intensity of untreated cells

### In Vivo and Tumor Studies

In this section, cell culture, tumor induction and drug injection studies were carried out. First, 20 μl of 1×10⁶ MDA-MB-231 human triple negative cancer cells were cultured in T75 tissue culture flasks (CELLTREAT, Pepperell, Mass., USA). This was carried out at 37° C. until 70% confluence was reached. The cells were grown under normal atmospheric pressure levels in a “L15′ medium” that is typically made up of: L-15 medium (ATCC, Manassas, Va., USA), supplemented with 100 I.U./ml penicillin/100 lg/ml streptomycin and 10% FBS (ATCC, Manassas, Va., USA).

Forty 4-weekold Athymic Nude-FoxnInu strain mice with individual weights of ˜17 g each was purchase from Envigo (Somerset, N.J., USA). These animals were approved for use in the current work by the University of Massachusetts Medical School Institutional Animal Care and Use Committee (UMMS IACUC) with docket #A2630-17. All of the animals were maintained and used according to the approved UMMS IACUC procedure and guideline.

Subcutaneous tumor xenografts were induced by the injection of 5.0×10⁶ of MDA-MB-231 human triple negative breast cancer cells (suspended in sterile saline) into the interscapular region (for a better angiogenic response). Tumor formation was carefully assessed by palpation. Tumor growth was then monitored daily with the digital calipers. The tumor volume was calculated using the following modified ellipsoidal formula:

$$Tumor Volume ( TV ) = \frac{{Width}^{2} \times Length}{2} \qquad ( 3 )$$

where length was the longest axis of the tumor and width is the measurement at a right angle to the length.

The mice were randomly chosen in groups of three (for each drugs injection) into their respective treatment groups. These include groups of mice with early stage (14 days after tumor induction), mid stage (21 days after tumor induction) and late stage (28 days after tumor induction) tumors. The weights of the mice and their tumor sizes were monitored and measured (using digital calipers) on a daily basis. These precise volumes and measured weights of the mice were used to guide the administration of the drugs. They were also used to monitor toxicity and side effects associated with the drugs. For each of the study groups, 3 mice each were randomly assigned to injection of 10 mg/kg of the specific drug configuration (PTX, [D-Lys6]LHRH-conjugated PTX and DMSO).

Different groups of mice were injected intravenously with each drug through their tail veins. This was done after tumor growth for 14, 21 and 28 days. The mice were injected with 10 mg/kg per week, during the two-week periods in which the effects of drugs were investigated. Following each drug administration, the tumor sizes were monitored with calipers on a daily basis (every 24 hours). In this way, the possible tumor shrinkage, growth or elimination, were monitored on a daily basis. Furthermore, the health status of the mice was monitored on a daily basis. This was done by monitoring the mice for signs of weight loss or altered motor ability in their cages. At the end of study, the mice were euthanized, following the approved IACUC guidelines and procedures. Thereafter, tumor tissues were excised from all of the mice, including tissues from their major organs (kidneys, spleen, liver and lungs).

### Histopathological and Toxicity Studies

Following the in vivo tumor induction and growth experiments, tissues were extracted from the kidneys, spleen, lungs, liver and tumor regions. These were immediately fixed in 4% paraformaldehyde, dehydrated in a graded series of alcohol, and embedded in paraffin. Double doses of 10 mg/kg of PTX and PTX-[D-Lys6]LHRH were then administered (on a weekly basis for two weeks) to female athymic nude mice that were subcutaneously-induced with TNBC. In this way, qualitative toxicity was studied by considering differences in mortality, changes in body weight, clinical signs, gross observations and the histopathology of the lungs, kidneys and the liver at different stages of tumor development. This was done for the different drugs and cancer treatment durations. Daily observations and weight measurements were also used to check for possible animal reactions to the drugs, physiological changes, weight loss/gain, and the general well-being of the mice.

Hematoxylin and eosin (H and E) staining was also carried out. This was used for the identification of tumor necrosis and the examination of histologic changes that occurred in vital organs, following the administration of the drugs. Briefly, formalin-fixed, paraffin-embedded tissue/organs (tumor, kidneys, liver and lungs) samples (5 m) were injected with PTX, [D-Lys6]LHRH-conjugated PTX drugs and DMSO. These were hydrated by passing them through decreasing concentrations (100, 90 &70%) of alcohol baths and water.

The hydrated tissue sections were then stained in hematoxylin solution for 5 mins. This was followed by rinsing with tap water for 3 minutes and differentiation in 1% acid alcohol for 5 minutes. Tap water was then used to rinse (three times) before dipping the sections in ammonia water for 2 minutes. This was followed by staining with eosin for 10 mins. The treated sliced samples were dehydrated in solution with increasing concentrations of alcohols followed by xylene. Finally, a few drops of Permount Mounting Medium were used to mount the resulting samples. The stained slides were then imaged with a 20× objective lens using a TS100F Nikon microscope (Nikon Instruments Inc., Melville, N.Y., USA) coupled with a DS-Fi3 C camera.

### Immunofluorescence Staining of Ligands-Conjugated Nanoparticles and Overexpressed Receptors

Immunofluorescence staining (IF) was used to characterize the overexpressed receptors on the triple negative breast cancer tissues. The IF was used to study the distributions of LHRH receptors that are over-expressed on the breast tumor.

In this section, frozen nude mice tissues were embedded slowly in optimum cutting temperature (OCT) compound. This was done in a cryostat (Leica CM3050 S Research Cryostat, Leica Biosystems Inc., Buffalo Grove, Ill., USA) to ensure that the tissues did not thaw. 10 m slices were obtained from specific frozen breast cancer tumors (obtained from the nude mice) that were then sectioned on a charged glass slides using a Leica cryomicrotome (Leica Biosystems Inc., Buffalo Grove, Ill., USA). The sliced sections were then allowed to dry overnight at room-temperature (˜23° C.) to enable them to adhere well to the glass slides for subsequent immunofluorescence staining. Following the adherence to glass slides, the sliced tumor samples were incubated with 0.5 ml of 3% bovine serum albumin (Sigma-Aldrich, St. Louis, Mo., USA) prepared with PBS mixed with 30 of triton X-100 (Life technologies Corporation, Carlsbad Calif.). This was done at room-temperature (˜23° C.) for 60 mins.

The blocking agents were then aspirated from the samples, which were then incubated with drop of 100 of anti-LHRH Antibody (Millipore Sigma, Burlington, Mass., USA) a primary antibody, to detect the levels of LHRH. This was done using a concentration of 1 g/ml in a desired dilution. The resulting samples were then incubated overnight at 4° C. before dip-rinsing three times (1 min each) in 1×PBS. The treated tumors were further incubated with 50 μl of anti-mouse IgG conjugated with Alexa fluoro 488 secondary antibody with concentration of 1 μg/mL for 2 hours. This secondary antibody was purchased from Thermo Fisher Scientific, Inc. (Waltham, Mass., USA). It was prepared at a concentration of 1 μg/ml in 1% BSA solution. The stained samples were then rinsed thrice in 10 ml 1× PBS for 1 min each.

Finally, the cell nuclei of the tumor samples were stained with drops of 5 μg/ml of ProLong Gold antifade reagent with DAPI (Thermo Fisher Scientific Inc., Waltham, Mass., USA). The resulting samples (on the glass slides) were fixed with coverslips using a few drops Permount Mounting Medium. The stained samples were then imaged at a magnification of 60× with Leica SP5 Point Scanning Confocal Microscope (Leica TCS SP5 Spectral Confocal couple with Inverted Leica DMI 6000 CS fluorescence microscope, Leica, Buffalo Grove, Ill., USA).

### Drug-Tissue Adhesion Study

In an effort to understand the specificity in the targeting of triple negative breast cancer via the receptors that are over-expressed on the tumor, adhesion measurements were carried out on the control xenograft tissue samples at different stages of tumor development. Adhesion forces and interactions (between the different drug molecules and receptors on the surfaces of the tumor tissues at different stages of development) were explored in an effort to understand the interactions of the drugs with the tumor and non-tumor tissue.

Antigen retrieval was carried out on the fixed tissue. This involved exposing target antigens to receptors on a 10 m thick microtome tissue slice. These sliced tissues were prepared for adhesion measurements in an Asylum MFP3D-Bio Atomic Force Microscope (AFM) (Asylum Research, Oxford Instrument, CA, USA). The AFM tips RESP-20 AFM tip (Bruker Santa Barbara, Calif., USA) were dip-coated with paclitaxel or [D-Lys6]LHRH-conjugated paclitaxel using the techniques described in Obayemi et al. (J. Mech. Behav. Biomed. Mater., 68 (2017), pp. 276-286).

A simple AFM tip dip-coating technique (J. D. Obayemi et al. Materials Science and Engineering C. 66, (2016), 51-65, Hampp, E. et al. Res. 27 (22), 2891, Hutter, J. L. et al. Instrum. 64, 1868) (of the drugs) was used to coat the AFM tips at room-temperature (˜23° C.). In addition, a positive control of LHRH peptides was coated onto the AFM tips and used to determine the adhesion forces between the receptors on breast cancer tissue. All of the coated AFM tips were air-dried for about 6 h and kept in a desiccator overnight before the adhesion measurements.

The spring constants of the coated and uncoated AFM tips were measured experimentally using the thermal tune method (J.D. Obayemi, et al. Mater., 68 (2017), 276-286). This was done to obtain the actual spring constants that were used to calculate the pull-off forces from Hooke's law. The adhesion interactions were measured for the following configurations of coatings on the AFM tips and breast cancer tumor at different stages on the mice:

(i) bare AFM tip to breast cancer tumor;
(ii) LHRH-coated AFM tip to breast cancer tumor;
(iii) LHRH-Paclitaxel coated AFM tip to breast cancer tumor; and
(iv) Paclitaxel coated AFM tip to breast cancer tumor.

### Statistical Analysis

In each case, an independent Student t test and one-way analysis of variance (ANOVA) were used to study the differences between the control and the study groups. A p-value<0.05 of significance was set.

### Results

### In Vitro Cell Viability and Inhibition

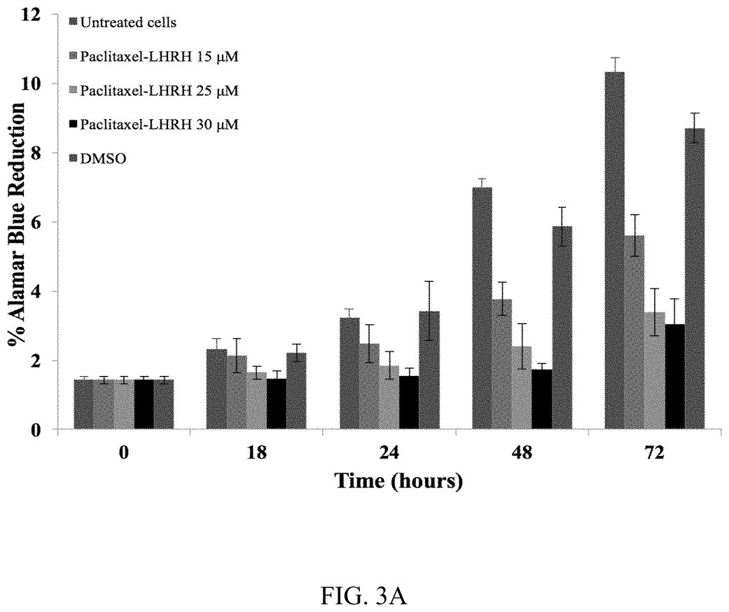

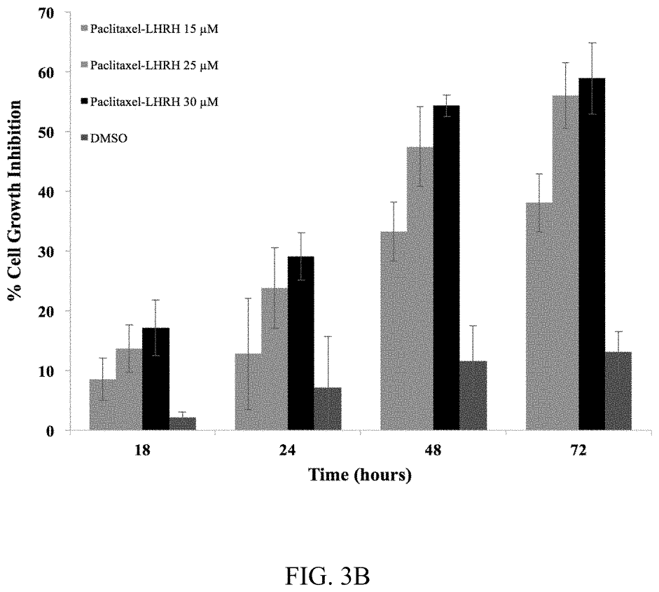

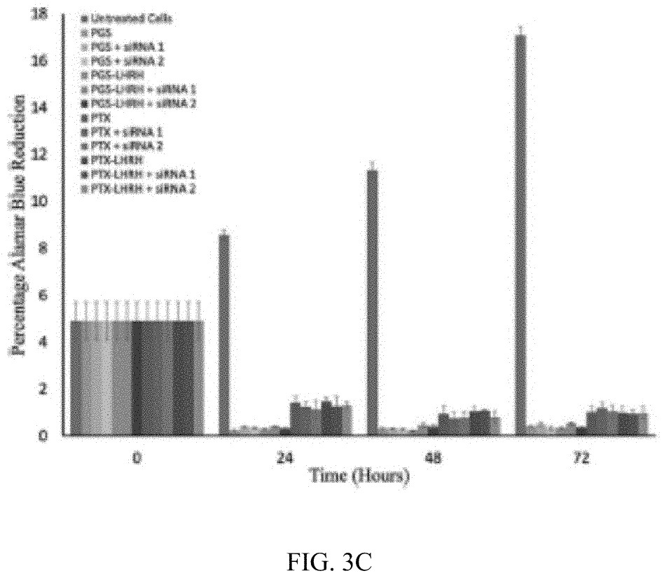

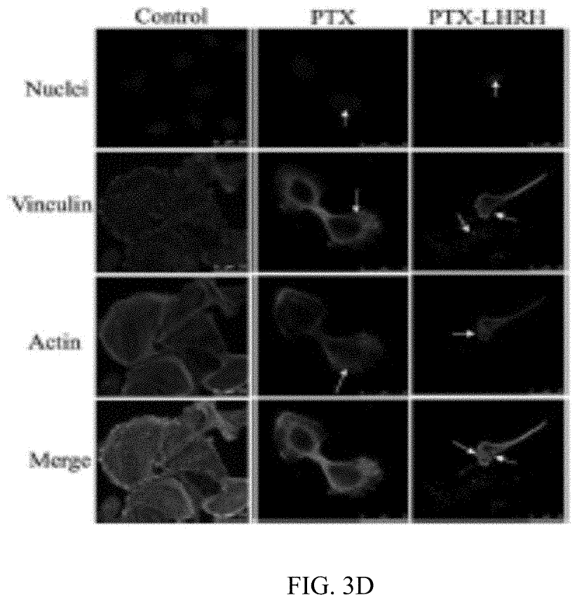

FIG. 3A compares the viability of untreated cells with those treated with drugs after 18, 24, 48 and 72 h of post-treatment. Among cells exposed to paclitaxel-[D-Lys6]LHRH drug and the DMSO control, it was found that increasing drug concentration had a greater effect on cell growth, as shown by the lower percentage alamar blue reduction values. Furthermore, by isolating the effect of DMSO alone (DMSO is the solvent used to dissolve the drugs), it was observed that there was no significant effect of DMSO on cell viability, when compared to that of [D-Lys6]LHRH-conjugated drugs. The assay revealed that the [D-Lys6]LHRH-conjugated PTX was more specific in their targeting of cancer cells.

The results presented in FIG. 3B show that the [D-Lys6]LHRH-conjugated PTX is effective at inhibiting the growth of MDA MB 231 cells. FIG. 3B shows a higher % inhibition values implies a higher cytotoxicity level due to drug-treatment. This trend increased with increasing drug concentration. Hence, the current results suggest that the [D-Lys6]LHRH-conjugated PTX is more specific in the targeting of the TNBC.

In the presence of the siRNA as shown in FIG. 3C, at times 24, 48 and 72 hours, there were no significant differences in cell viability between PTX and LHRH-conjugated PTX when the cells were treated with the siRNAs. Consequently, the unconjugated and LHRH-conjugated drugs exhibited similar anti-proliferative effects on the cells due to the suppression of LHRH receptor-mediated drug entry into the cells. Without cell treatment with siRNA, the results in FIG. 3A showed that the LHRH-conjugated drugs significantly reduced cell viability than the unconjugated drugs due to the specific targeting of the cells. This result is attributed to the specific interactions between the LHRH and the LHRH receptors in the absence of the knock down, and the reduced access of the conjugated or conjugated drugs after the knock down of the cell LHRH receptors by the siRNA.

Furthermore, from the confocal fluorescence images of drug-interacted cells (FIG. 3D), it is clear that treatment with the drugs result in the degradation, disorganization and depolymerization of the actin filaments and vinculin structures. The drugs also disrupted the cancer cell membranes and cytoskeletal actin structures. These disruption and disintegration give rise to apoptosis and cell death. This phenomenon was more evident in LHRH conjugated drugs (LHRH-PTX) than unconjugated drugs (PTX). In general, the current results show that the conjugation of the cancer drugs to the LHRH peptide increases the selectivity, effectiveness, and uptake of anticancer drugs to TNBC, due to the presence of overexpressed LHRH receptors on the surfaces of the TNBC.

### In Vivo Tumor Development and Shrinkage

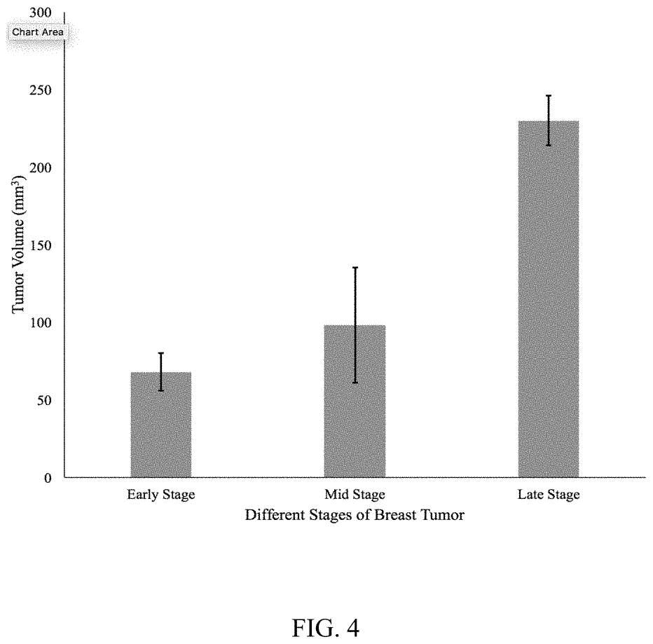

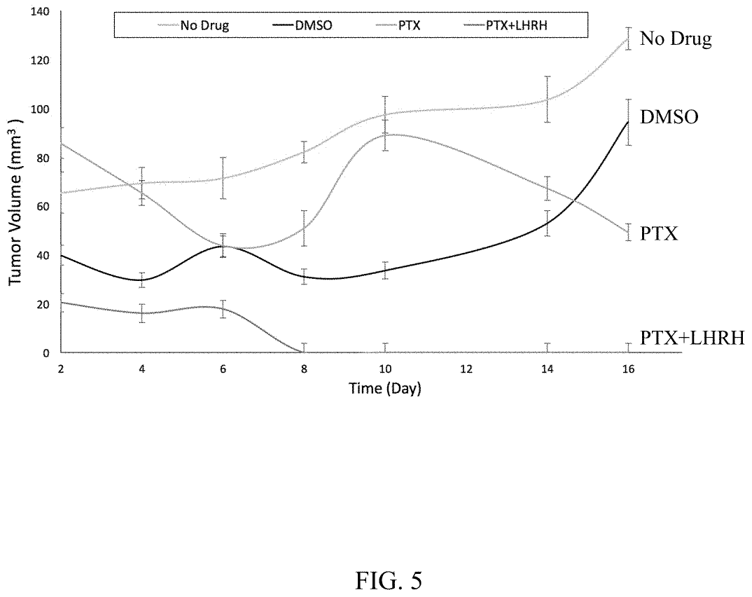

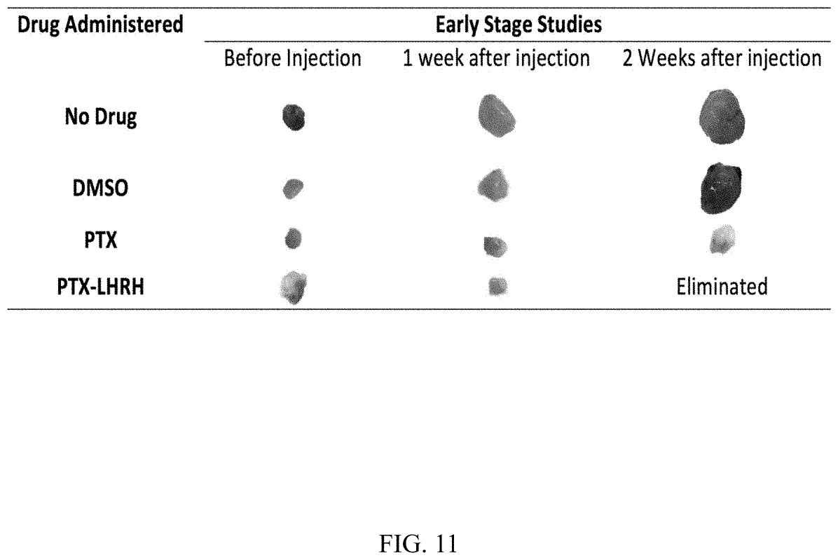

The mean tumor volumes for the mice before treatment on day 14, day 21 and day 28 were 67 mm³, 98 mm³ and 230 mm³, respectively (FIG. 4). In the case of the day 14 group, tumor elimination was observed two weeks after the injection of [D-Lys6]LHRH-conjugated PTX. The initial tumors in the mice were eliminated after administering two injections (one per week) of 10 mg/kg (each) of [D-Lys6]LHRH-conjugated PTX (FIG. 5 and FIG. 11). This is in contrast to the unconjugated PTX drug that resulted in some tumor shrinkage and final tumor sizes of ˜49.1 mm³.

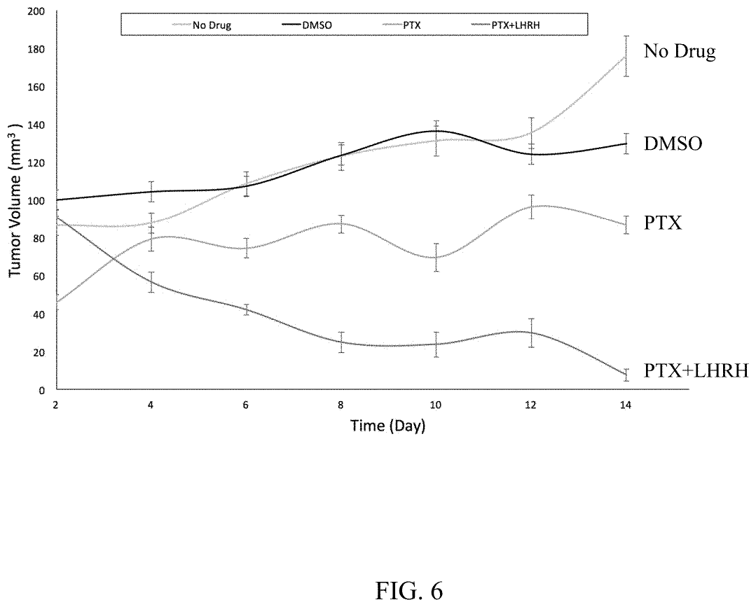

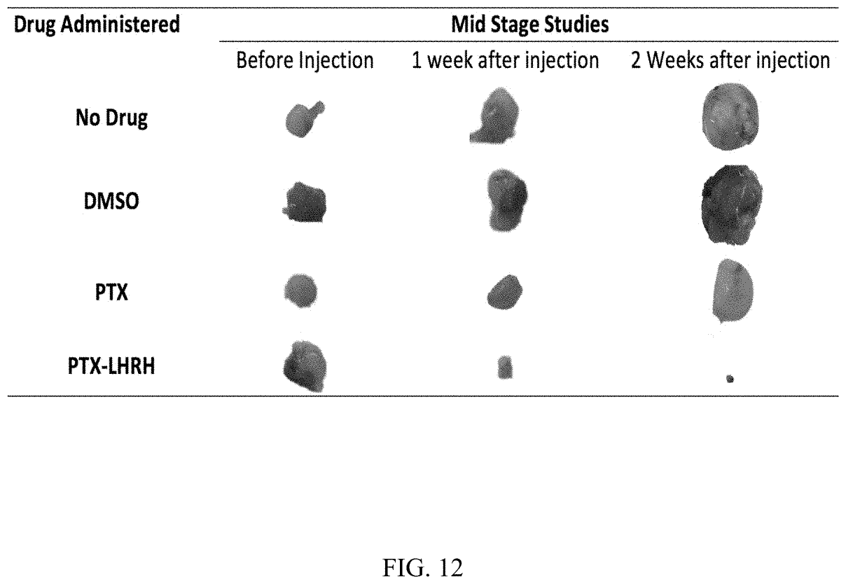

In the case of the 21-day group treatment, significant shrinkage was observed after about two weeks of administration of [D-Lys6]LHRH-conjugated PTX, when compared to that associated with PTX. These resulting tumor volume (FIG. 6 and FIG. 12) associated with the PTX-[D-Lys6]LHRH was 7.76 mm³. These are much smaller than the tumor volumes associated with treatment with the non-conjugated PTX, which resulted in tumor volumes of 86.83 mm³. This implies that there was 91% decrease in the xenograft volume after the administration of [D-Lys6]LHRH-conjugated PTX, compared to that associated with unconjugated PTX.

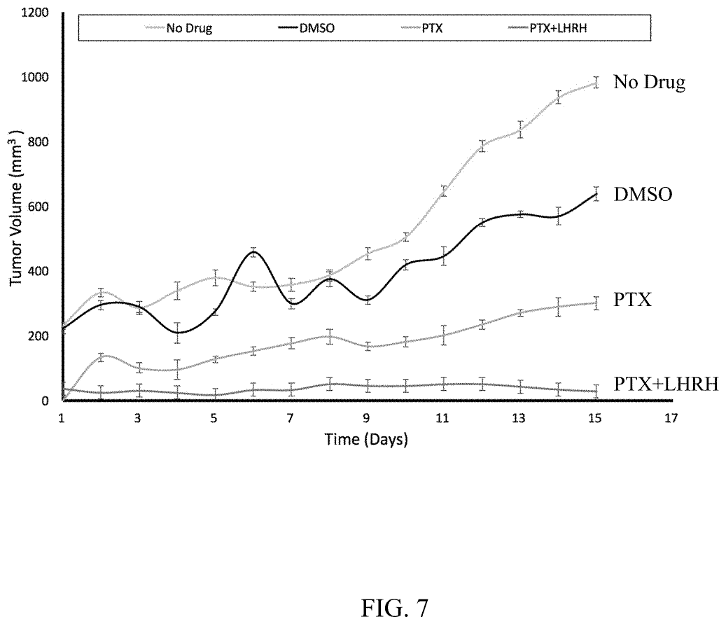

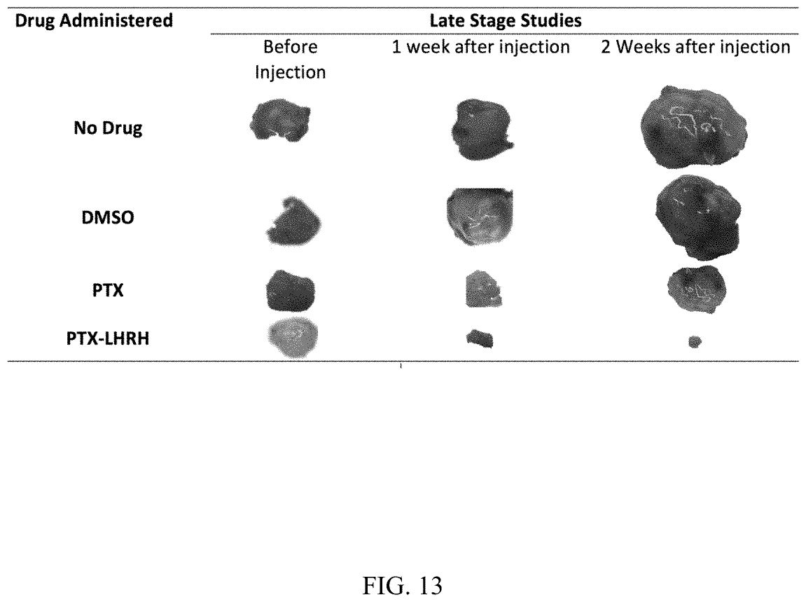

In the case of the 28-day treatment, significant tumor shrinkages were observed in the xenograft tumor sizes (FIG. 7 and FIG. 13) during the two weeks of drug administration (29.4 mm³ for PTX-[D-Lys6]LHRH), as compared to 299.2 mm³ for the unconjugated PTX drug. The percentage reduction in xenograft tumor volume for [D-Lys6]LHRH-conjugated PTX was 90.2%, as compared to the unconjugated PTX drug. The above results show that in each of the treatment groups (14-day, 21-day and 28-day), the use of [D-Lys6]LHRH-conjugated PTX exhibited significant anti-tumor effects.

### Ex Vivo Immunofluorescence Staining and Adhesion Measurements

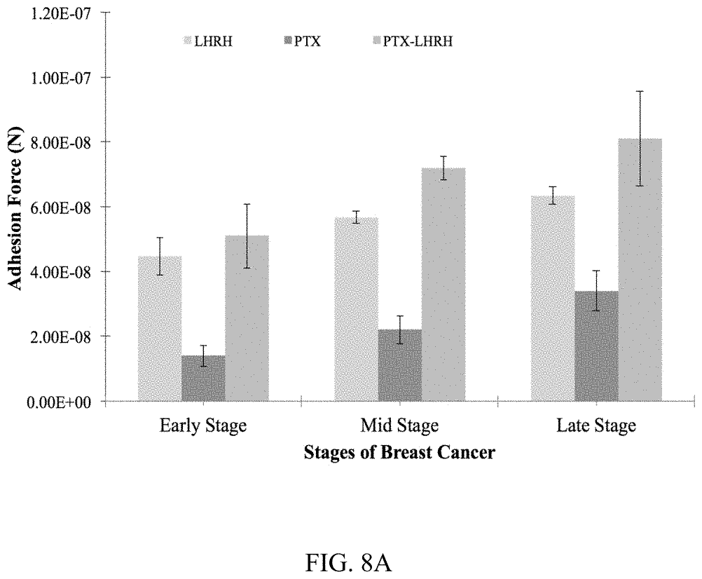

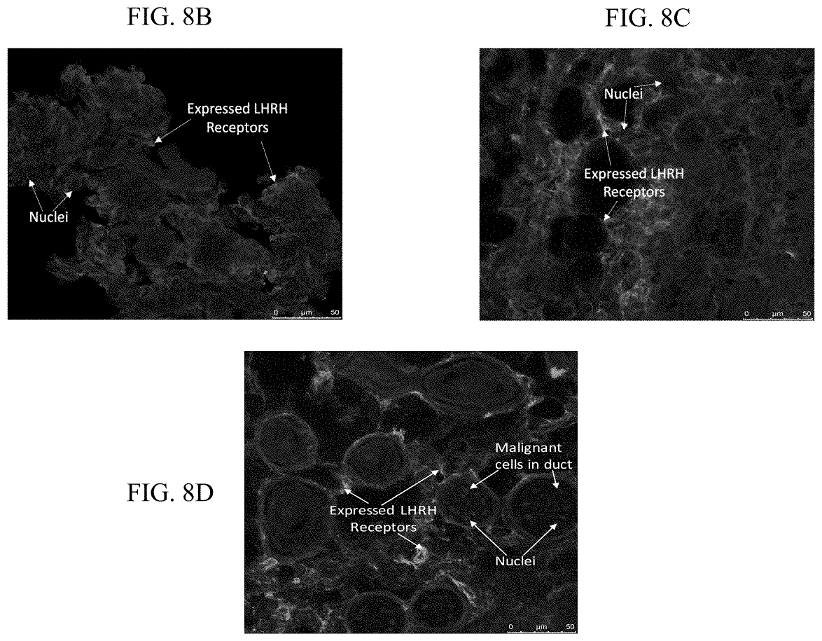

In FIG. 8A, the adhesion results show that adhesion forces/interaction between the LHRH-conjugated drug molecule increases with the stages of breast cancer tumor. This was seen in the immunofluorescence staining (FIG. 8B-D) as the densities of LHRH receptors increase from the early to the late stage of the breast cancer tumor. Relatively low adhesion forces (14 nm, 22 nm and 34 nm) were obtained between the unconjugated PTX, and the respective breast tumors in the early stage, mid stage and late stage conditions. However, in the case of [D-Lys6]LHRH-conjugated PTX, higher average adhesion forces 51 nm, 72 nm, 81 nm) were obtained for early stage, mid stage and late stage tumors compared to those in the unconjugated drugs.

The above results suggest that the highest therapeutic activity was associated with the [D-Lys6]LHRH-conjugated PTX (FIGS. 5-7). Also, for xenograft tumors that were induced subcutaneously at the intrascapular sites, the intravenous injection of [D-Lys6]LHRH-conjugated PTX via the tail vein shrunk or eliminate the induced tumor at different stages of tumor development (FIGS. 5-7). The [D-Lys6]LHRH-conjugated paclitaxel drug, therefore, enhanced the specific targeting of TNBC in the athymic nude mouse model that was examine in this study. The side effects associated with the specific delivery of these drug were also minimal.

Also, for xenograft tumors that were induced subcutaneously at the intrascapular sites, the intravenous injection of [D-Lys6]LHRH-conjugated PTX via the tail vein shrunk or eliminate the induced tumor at different stages of tumor development (FIGS. 5-7). The [D-Lys6]LHRH-conjugated paclitaxel drug, therefore, enhanced the specific targeting of TNBC in the athymic nude mouse model that was examine in this study. The side effects associated with the specific delivery of these drug were also minimal.

The injection of 10 mg/kg of [D-Lys6]LHRH-conjugated paclitaxel eliminated the tumors that were formed within the early stages of tumor development (within 14 days), without any evidence of toxicity (FIG. 5). The same concentration of drug also resulted in significant shrinkage of the mid- and late-stage tumors that were formed after 21 and 28 days, without any toxicity (FIGS. 6-7). This suggests that extended treatments (beyond the two-week injection period that was explored in this study) could result in the elimination of mid- and late-stage tumors. The results obtained from the adhesion measurements and immunofluorescence staining also show that improved therapeutic effects of the LHRH are associated with the increased adhesion of LHRH-conjugated cancer drugs ([D-Lys6]LHRH-PTX) to LHRH receptors that are overexpressed on the surfaces of triple negative breast cancer cells.

The improved therapeutic effects of the LHRH-conjugated drugs are also associated with the increase adhesion of LHRH-conjugated drugs to the LHRH-receptors that are shown to be overexpressed on the surfaces of the tumor tissue (FIGS. 8B-D).

In general, the average adhesion forces between the [D-Lys6]LHRH-conjugated PTX was nearly three times that of unconjugated PTX to the early stage breast tumor. In the case of the mid stage breast tumor, the adhesion force of [D-Lys6]LHRH-conjugated PTX is more than three times for those of PTX drug. For the late stage tumor, the adhesion force [D-Lys6]LHRH-conjugated PTX was about 2 times for those of PTX drug (See FIG. 8A). The increase in adhesion force is attributed to increased incidence of LHRH receptors on the surfaces of the breast tumors. These give rise to increased adhesion via hydrogen bonding and van der Waals interactions between the conjugated drugs and TNBC tissue.

### Histopathology and Toxicity

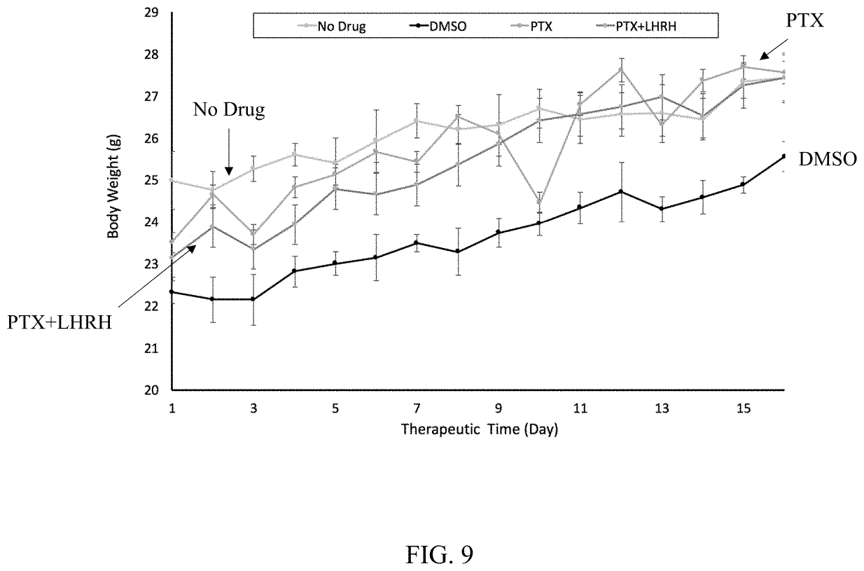

The tumor growth rates associated with the therapeutic period are presented in FIG. 9. This shows that there were no significant changes in the body weight associated with all of the dosing groups tested. Furthermore, there were no significant physiological changes, clinical signs, changes in mortality, or changes in the body weight after the administration of the drugs, compared to the control mice. The body weight measured during the therapeutic period corresponds to the body weight ranges of same aged normal mice in all of the tested groups, including control mice. All of the mice appeared to be healthy with normal eyes, fur and skin conditions, during the 14 days of treatment and observation.

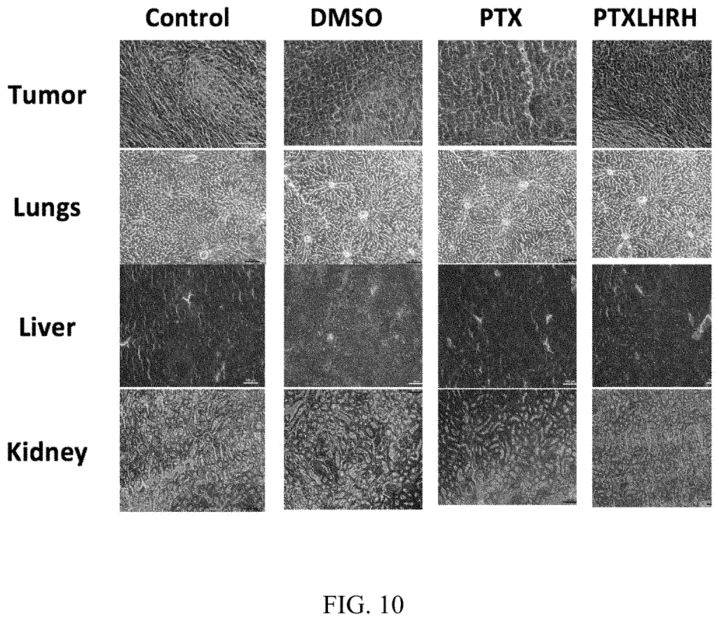

Histopathological examination of tumor tissue showed that tumor cells from the PTX-[D-Lys6]LHRH treated mice exhibited disorder and different sizes. They also appear to be more mitotic. The images presented in FIG. 10 shows the structure of the tumor tissue extracted from the xenograft breast models after treatment with LHRH-conjugated and unconjugated drugs. The stained images reveal evidence of increased angiogenesis as a result of fibrous necrosis in the tumor tissues. Treatment with [D-Lys6]LHRH-conjugated PTX resulted in higher levels of necrosis in the tumors, when compared to those in the animals treated with the unconjugated PTX drug.

The toxicities associated with the injected drugs were also verified using H&E staining. The results showed that were no significant histological or significant pathological changes in the liver, lung, and kidneys of the mice that were treated with [D-Lys6]LHRH-conjugated PTX or unconjugated PTX injected mice. Hence, the features observed in these mice were comparable to those in as the control mice organs.

In the case of the [D-Lys6]LHRH-conjugated PTX groups, there was no evidence of liver cell hyaline degeneration and necrosis, and no pulmonary edema or hyperplasia observed in the lungs. There was also no evidence of hyperplasia, and the glomerular volume of the kidneys was normal. Furthermore, no chemotherapeutic drug-induced histological changes and tumor metastasis were observed in the [D-Lys6]LHRH-conjugated PTX groups. Hence, the observed shrinkage or elimination of tumors was associated with the targeted [D-Lys6]LHRH-PTX drug and did not induce any degeneration in the primary organs such as kidneys, liver and lungs.

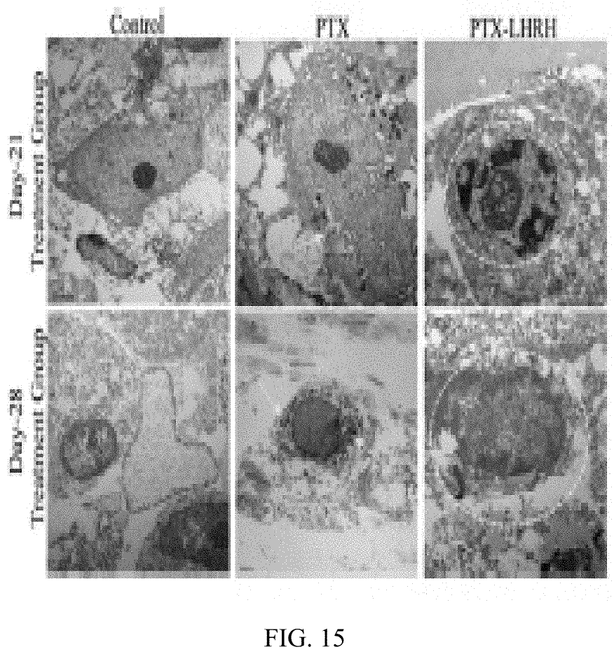

FIG. 15 presents TEM images of the drug treated tumors obtained from the 21-day and 28-day treatment groups. The TEM images revealed evidence of greater structural changes in the cancer cells/tissues injected with LHRH-PTX than in those injected with PTX. The circled and pointed structures observed are changes in the structure of the membranes and nuclei are attributed to the effects of the drugs on the tumor tissue. The structural changes in the breast cancer tissues are attributed to due to drug effects on the breast cancer tissues. These include shrinkage and the disorganization of the nuclei (nuclear fragmentation) and the cell membranes that are revealed in the images of the breast cancer tissues that were obtained from animals that were treated with the conjugated drugs.

LHRH Receptors Staining, siRNA Knockdown, RT-qPCR Quantification

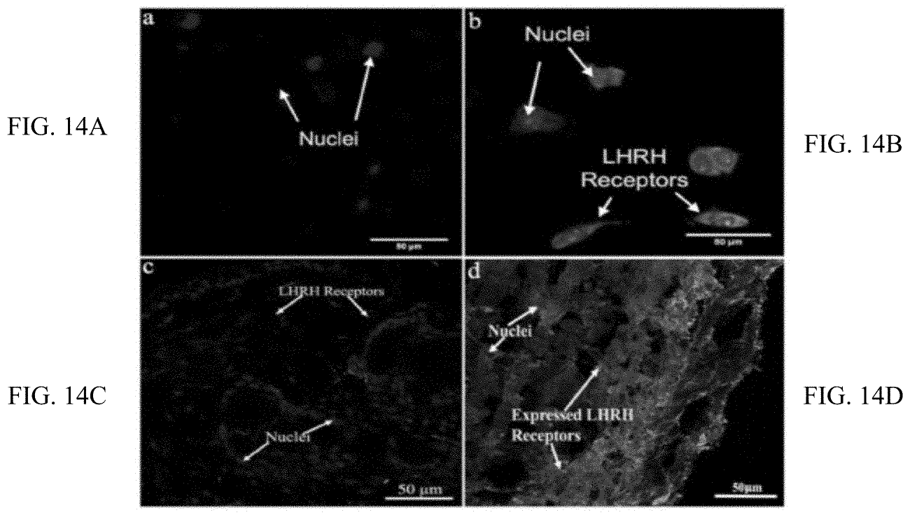

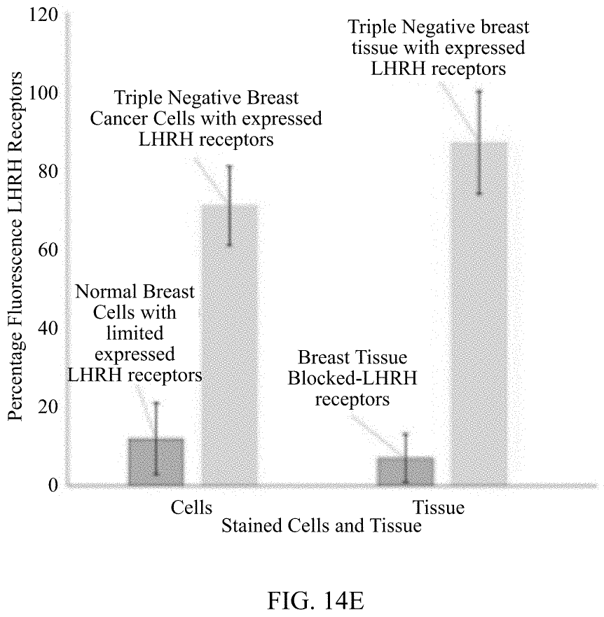

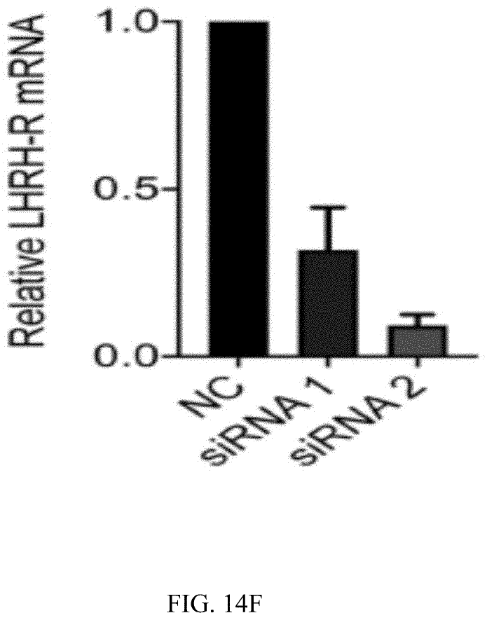

FIGS. 14A and 14B show expression of LHRH receptors (green stain) on non-tumorigenic epithelial breast cell line (MCF 10 A) compared to those of triple negative breast cancer cells (MDA MB 231) via immunofluorescence staining. Results showed that evidence of LHRH receptors on TNBC.

In a similar fashion, LHRH receptors are seen to be overexpressed on unblocked LHRH antibody receptors stained TNBC tissue. In the case of blocked LHRH TNBC cells, the receptor expression obtain from fluorescence confocal microscope was very low (FIG. 14C) as compared to those that were unblocked (FIG. 14D). In both cases (FIGS. 14A-14D), the percentage fluorescence LHRH receptors was quantified as shown in FIG. 14E. These results provide evidence of expression of LHRH receptors on TNBC. Furthermore, results from the knock down experiment using two sets of siRNA show that it knocked down the LHRH receptor in MDA-MB-231 cells and observed a ˜70% and 90% reduction of LHRH receptor transcript levels (FIG. 14F). Knockdown of LHRH receptor significantly reduces the enhanced delivery of PTX achieved by LHRH peptide conjugation.

### Example 2: Encapsulated LHRH-Paclitaxel Conjugates

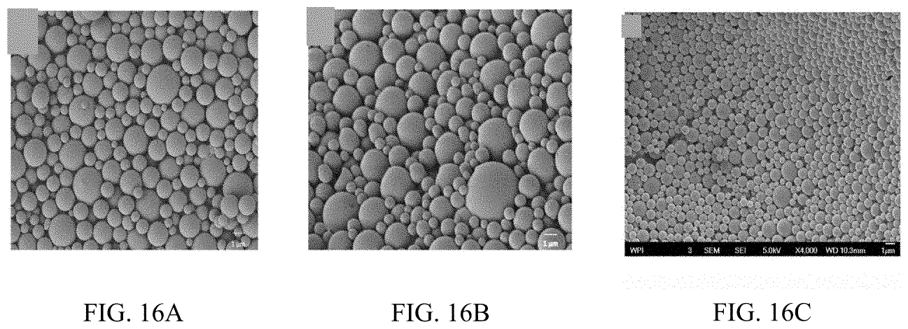

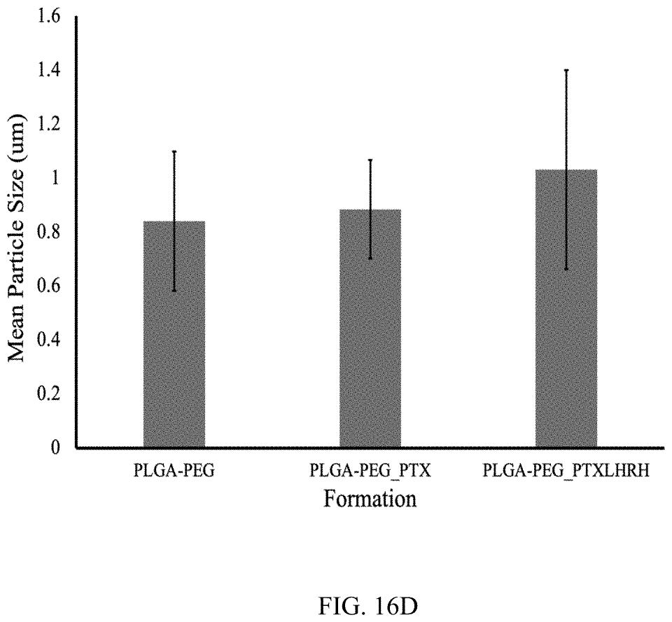

Microparticle characterization. SEM images of the polymer blend drug-loaded microspheres with their and control microspheres are presented in FIGS. 16A-16C. Our results show that there are no significant morphological differences between the drug-loaded PLGA-PEG microspheres and the control PLGA-PEG microspheres. This suggests that the presence of drug did not significantly affect the morphologies of the drug-loaded micro-spheres. Furthermore, the mean particle sizes of the microparticles were between 0.84 and 1.23 m (FIG. 16D). The hydrodynamic diameter obtained from the DLS (Table 1) were greater than the mean diameter obtained from the SEM (FIG. 16D). This could be attributed to the PEG being soluble in the DLS medium leading to a swollen structure with high water content.

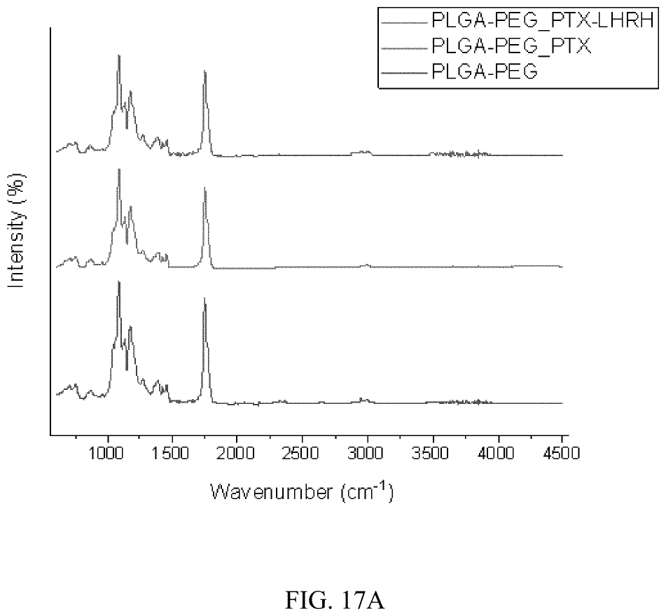

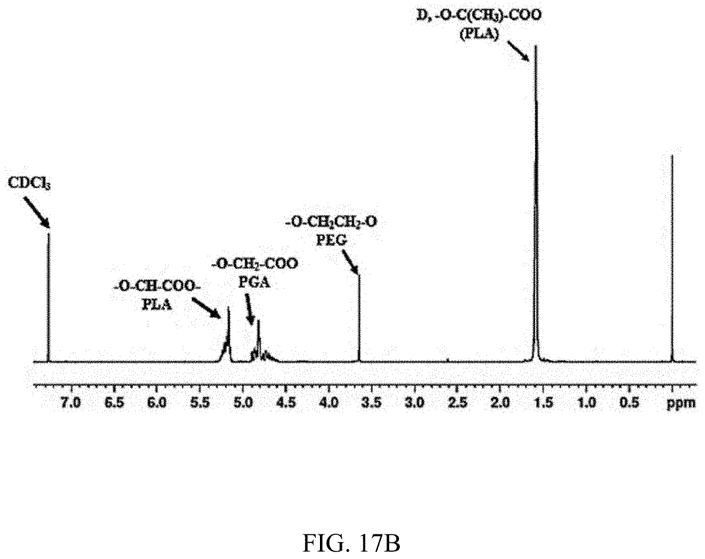

The FTIR spectra obtained for the drug-loaded PLGA-PEG microspheres were similar to those of the control PLGA-PEG microspheres (FIG. 17A). This indicates that there was no significant modification on the chemical groups of PLGA and PEG due to drug loading. Hence, in each case, the characteristic peaks that were obtained for PLGA and the PEG polymer. These were present before and after drug loading. Thus, the FTIR spectra obtained for the drug-loaded and control PLGA-PEG microspheres showed a strong band at 1749 cm⁻¹. This corresponds to the C═O stretch in the lactide and glycolide structure. A characteristic peak of PEG was revealed at 1,084 cm⁻¹. This is equivalent to the C—O stretch. The identical FTIR spectra of the conjugated drug-loaded microspheres correspond to those of the spectrum of the blend of polymer (PLGA-PEG). Results from the drug-loaded spectra show the absence of characteristic intense bands of the drugs used (PTX, PTXLHRH). In each case, the absence of the peaks may have been masked by the bands produced by the blend of polymer. This result suggests the presence of drugs as a molecular dispersion in the blend polymer matrix due to the absence of chemical interaction between the blend of polymer (PLGA-PEG).

TABLE 1
The mean diameter (SEM), the hydrodynamic hydrometer (DLS)
and the polydispersity index (PDI) values for the various
PLGA-PEG microspheres formulations.

| Formulation | SEM (μm) | DLS (μm) | PDI |
|---|---|---|---|
| PLGA-PEG | 0.80 ± 0.26 | 3.14 ± 0.09 | 0.82 |
| PLGA-PEG-PTX | 0.88 ± 0.18 | 5.26 ± 0.53 | 0.58 |
| PLGA-PEG-LHRH-PTX | 1.03 ± 0.37 | 6.02 ± 0.80 | 0.39 |

Similar HNMR spectra were obtained for all of the PLGA-PEG microsphere formulations, with four sets of principal peaks (ppm). FIG. 17B shows representative HNMR spectra for the different formulations of PLGA-PEG microspheres. The peak at 3.64 ppm corresponds to the hydrogen atoms in the methylene groups of the PEG moiety. Hydrogen atoms in the methyl groups of the d- and 1-lactic acid repeat units resonated at 1.57 ppm with an overlapping pair. A highly complex peak, due to several different glycolic acid, d-lactic, 1-lactic sequences in the polymer backbone, was observed at 4.81 ppm and 5.20 ppm. This corresponds to the glycolic acid CH₂ and the lactic acid CH, respectively. Deuterated chloroform was used as a solvent and a chemical shit was seen at 7.26 ppm. These results suggest that the blend of polymers did not undergo chemical modification during drug loading and encapsulation.

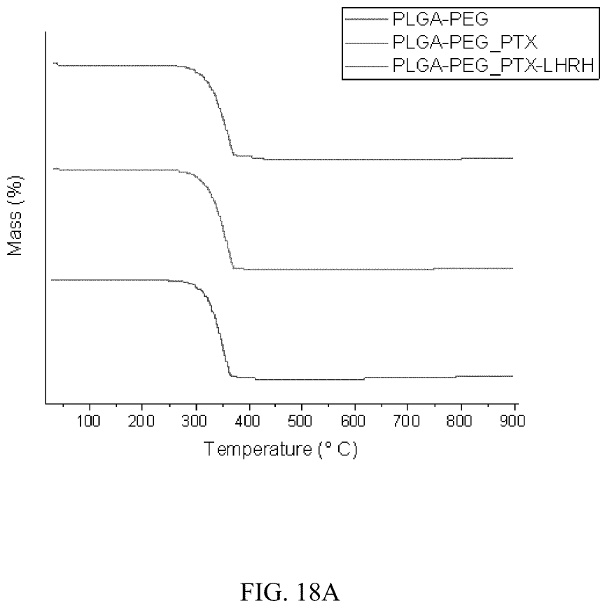

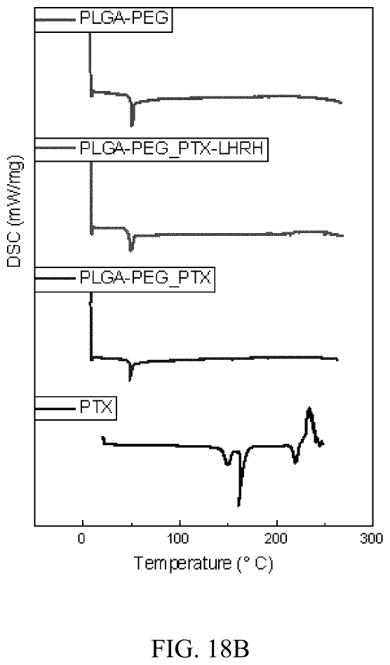

FIG. 18A and FIG. 18B show the thermal decomposition process of control PLGA-PEG microspheres and drug-loaded PLGA-PEG microspheres obtained via Thermogravimetric Analysis (TGA). The TGA thermograms reveal one stage of weight loss. This suggests that the polymers and respective drugs mix but do not interact. The one step decomposition in the TGA analysis (FIG. 18A) may be due to the decomposition of the PLGA moiety in the blend64. The decomposition temperatures of the control PLGA-PEG microspheres and the drug-loaded PLGA-PEG microspheres are presented in FIG. 18B. The results show that the decomposition temperature decreases with drug loading.

The DSC thermograms are presented in FIG. 18B. This reveals that the control PLGA-PEG microspheres and drug-loaded PLGA-PEG microspheres exhibited similar endothermic events with a single defined peak. This suggests that the drug-loading did not affect the polymer structure. In the case of the control PLGA-PEG micro-spheres, the glass transition temperature (T_{g}) and the melting temperature (Tₘ) were measured to be 48.3° C. and 51.3° C., respectively (Table 2). The ΔCp corresponds to 0.411 J/(g K). However, in the case of drug-loaded PLGA-PEG microspheres, the T_{g} and Tₘ were lower than those of the control PLGA-PEG microspheres, leading to higher ΔCp values. These changes in the measured values are attributed to the effects of the respective drugs, which act as a plasticizers for the polymer (PLGA).

TABLE 2
The Glass transition temperature (Tg), Endothermic peak and Delta Heat Capacity
(ΔCp) values for the various PLGA-PEG microspheres formulations.

| | Glass transition | | | |
|---|---|---|---|---|
| Drug-loaded | temperature (Tg) | Endothermic peak | Delta heat capacity | Decomposition |
| composition | (° C.) | (° C.) | (ΔCp) J/(g K) | temperature (° C.) |
| PLGA-PEG | 48.3 | 51.3 | 0.411 | 334.4 |
| PLGA-PEG-PTX | 47.3 | 49.6 | 0.495 | 330.5 |
| PLGA-PEG-LHRH- | 47.6 | 50.1 | 0.479 | 325.7 |
| PTX | | | | |

Furthermore, it was also observed that crystalline PTX had an endothermic peak corresponding to a melting point of 220° C. It should be noted that due to the concentration and the very low drug loading of the drug in the respective microspheres, there was no any noticeable signature peaks of corresponding drug formed in each drug-loaded system. This result indicate that each drug encapsulated did not crystallize in the blend of polymer microspheres. Generally, it was observed that the encapsulation of drug into the polymer microspheres did not significantly change the thermal properties of the drug-loaded polymer systems.

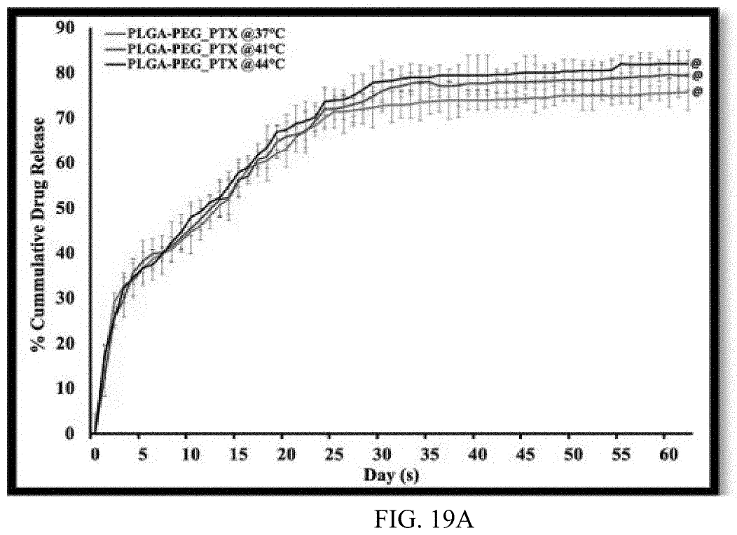

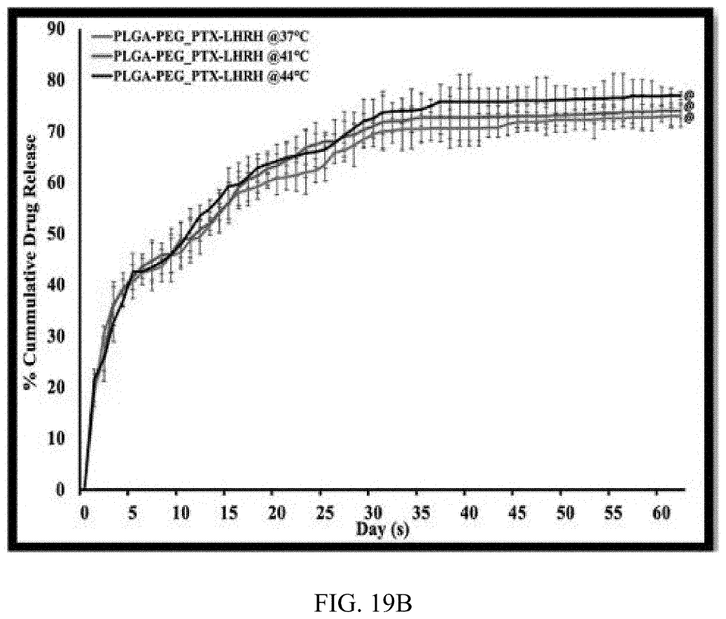

In vitro drug release. FIGS. 19A-19B show the time dependence of the percentage of cumulative drug release from the drug-loaded PLGA-PEG microspheres. All of the drug-loaded formulations revealed similar release profiles.

After 62 days, ˜80% of PTX and LHRH-PTX drugs was released. Finally, in this section, it is important to note that controlled release occurred from the microspheres (with ˜60% release) within ˜40 days. The respective drug encapsulation efficiencies and their drug loading efficiency obtained for the drug-loaded microspheres (PLGA-PEG_PLGA-PEG-PTX, PLGA-PEG-LHRH-PTX), were determined to be ˜72%, 38% and 16.1%, 9.8%, respectively. In each case of the drug release studies, the results were not significant since the p value for each drug at different temperatures considered are greater than 0.05. This implies that there was no significant difference when different temperatures were used. However, com-paring the respective cumulative drug release, the results were considered to be significant with a p value <0.05.

Drug release kinetics. The drug release kinetics (Table 3) obtained from the drug release data that were fitted in the kinetic models [zero order (Qₜ=Q₀+K₀·t), first order (log Qₜ=log Q₀+Kt/2.303), Higuchi model (Qₜ=K_{H}·t^{1/2}) and Korsmeyer-Peppas model

$$( \frac{M t}{M ∞} = {Kt}^{n} ) ]$$

showed that the Korsmeyer-Peppas model provided the best fit to the experimental data obtained for the different drug-loaded PLGA-PEG microsphere formulations. In some cases, the release exponent ‘n’ was between 0.446 and 0.889, which is consistent with drug release by anomalous transport or non-Fickian diffusion that involves two phenomena: drug diffusion and relaxation of the polymer matrix.

TABLE 3
The kinetic constant (K), correlation coefficient (R²) and Release exponent (n) of
kinetic data analysis of drug released from the various PLGA-PEG microspheres formulations.

| | Temperature | Zero order | | First order | | Higuchi model | | Koresmeyer-Peppas | | |
|---|---|---|---|---|---|---|---|---|---|---|
| Formulations | (° C.) | K | R² | K | R² | K | R² | K | R² | n |
| PLGA-PEG- | 37 | 0.769 | 0.692 | 0.008 | 0.330 | 8.137 | 0.867 | 3.271 | 0.962 | 0.459 |
| PTX | | | | | | | | | | |
| PLGA-PEG- | | 0.680 | 0.704 | 0.007 | 0.294 | 7.802 | 0.845 | 3.340 | 0.848 | 0.490 |
| LHRH-PTX | | | | | | | | | | |
| PLGA-PEG- | 41 | 0.853 | 0.718 | 0.009 | 0.354 | 8.964 | 0.886 | 3.398 | 0.969 | 0.447 |
| PTX | | | | | | | | | | |
| PLGA-PEG- | | 0.685 | 0.672 | 0.007 | 0.288 | 7.316 | 0.856 | 3.431 | 0.912 | 0.446 |
| LHRH-PTX | | | | | | | | | | |
| PLGA-PEG- | 44 | 0.881 | 0.728 | 0.009 | 0.357 | 9.224 | 0.951 | 3.210 | 0.985 | 0.490 |
| PTX | | | | | | | | | | |
| PLGA-PEG- | | 0.753 | 0.712 | 0.008 | 0.311 | 7.939 | 0.885 | 3.302 | 0.968 | 0.450 |
| LHRH-PTX | | | | | | | | | | |

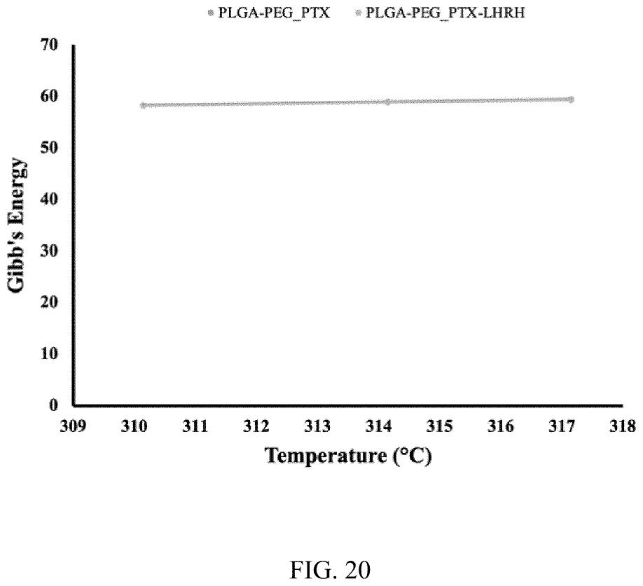

Thermodynamics of drug release. The thermodynamic parameters (ΔG, ΔH, ΔS and Eₐ) that were obtained from this study are presented in Table 4. The change in the Gibb's free energy (ΔG) was negative for all of the PLGA-PEG microsphere formulations. This indicates the feasibility and non-spontaneous nature of the drug release from the PLGA-PEG microspheres at all temperatures. FIG. 20 shows a plot of Gibb's free energy versus Temperature for various PLGA-PEG formulations. The negative values obtained for the change in entropy (ΔS) also confirm that there is a decrease in the disorder associated with drug release from the various PLGA-PEG microspheres. Furthermore, the positive values obtained for the change in enthalpy (ΔH) confirm that the drug release process (from all of the PLGA-PEG microspheres formulations containing) was endothermic. However, a positive Eₐ was obtained for the drug release from all the PLGA-PEG formulations, indicating that in all cases, the rate of drug release increased with increasing temperature.

TABLE 4
Thermodynamic parameters for the various PLGA-PEG microspheres.

| | Temperature | E a | ΔS (kJ mol−¹ | | |
|---|---|---|---|---|---|
| Formulations | (° C./K) | (kJ mol−¹) | K−¹) | ΔH (kJ mol−¹) | ΔG (kJ mol−¹) |
| PLGA-PEG-PTX | 37/310.15 | 7.714 | −0.163 | 7.714 | 58.268 |
| | 41/314.15 | | | | 58.920 |
| | 44/317.15 | | | | 59.409 |
| PLGA-PEG-LHRH-PTX | 37/310.15 | 5.444 | −0.170 | 5.444 | 58.170 |
| | 41/314.15 | | | | 58.850 |
| | 44/317.15 | | | | 59.360 |

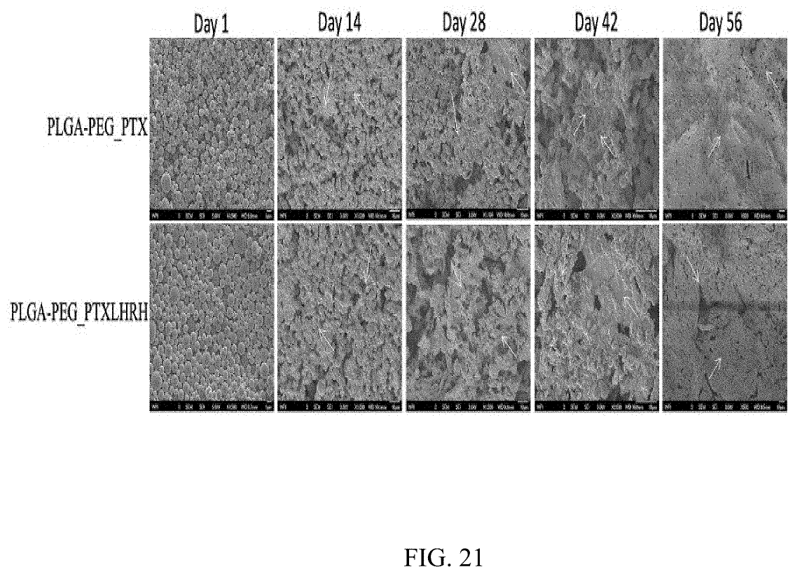

Degradation of drug-loaded microspheres. SEM images of the degradation of the drug-loaded micro-spheres are presented in FIG. 21. Gradual morphological changes were observed within the 56-day period of the drug release experiments. After 24 h of exposure to the release medium (PBS, pH 7.4), the surfaces of the drug-loaded PLGA-PEG microspheres were still smooth with micropores. However, by day 14, morphological changes were observed. These included microsphere agglomeration, distinct micropores and less spherical shapes. Evidence of microsphere agglomeration and void formation was observed by Day 28. After 42 days of drug elution, the surfaces of the PLGA-PEG microspheres were completely eroded visibly larger pores. Further evidence of material removal was also observed after 56 days of drug elution, which was found to result in more porous structures than those that were observed before drug elution. The increased erosion is attributed to the hydrolytic degradation of the ester and drug leaching.

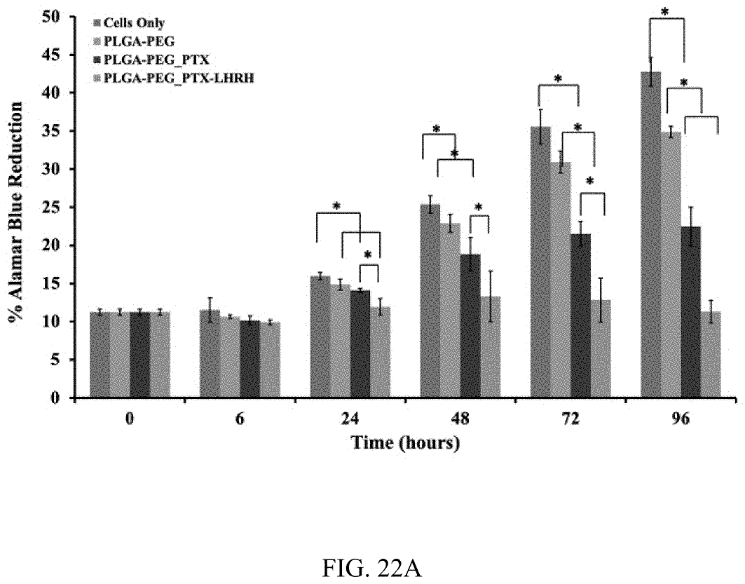

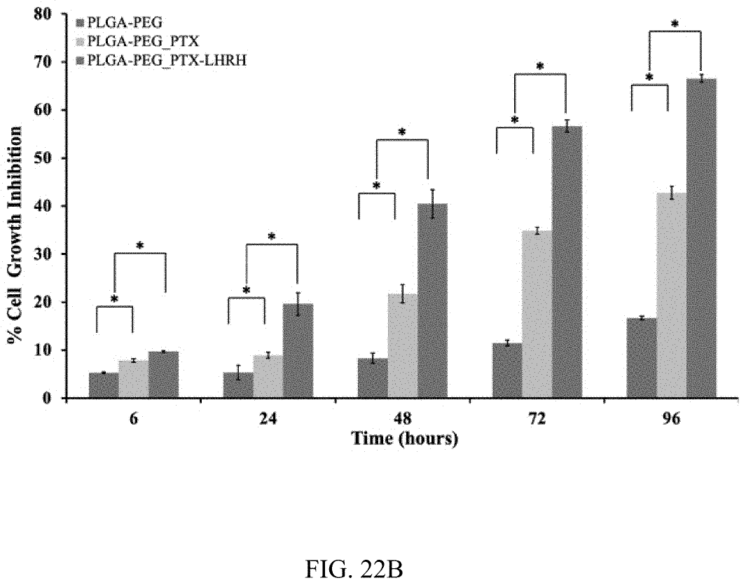

Cell culture. In vitro cell viability and drug cytotoxicity. FIG. 22A and FIG. 22B compares the percentage alamar blue reduction and percentage cell growth inhibition, respectively, for cells only (MDA-MB-231 cells), drug-loaded and control PLGA-PEG microspheres 6, 24, 48, 72 and 96 h post-treatment. The percentage alamar blue reduction measures the cell metabolic activity, which is a function of the cell viability and cell population. This implies that a higher percentage of alamar blue reduction value corresponds to a higher cell growth and, by extension, a higher cell viability. A two-way ANOVA with post hoc Tukey HSD multiple comparisons tests showed that, generally, the cell viability was significantly lower (p<0.05) for the cells treated with drug-loaded PLGA-PEG microspheres than cells that were not exposed to drug elution from microspheres. Furthermore, the cells treated with PLGA-PEG microspheres loaded with conjugated drugs were less viable than their counterparts that were loaded with unconjugated drugs. This means that the conjugated drugs were more effective at reducing the metabolic activities of the MDA-MB-231 cells than their unconjugated counterparts. The statistically significant group pairs of interest (p<0.05) are highlighted.

There was a slight reduction in cell viability when the cells were exposed to the control PLGA-PEG micro-spheres (no drugs), attributed to the cytotoxic effects of leached residual DCM solvent that was used to process the microspheres. However, the reduction in cell viabilities ((FIG. 22A) and increase in cell growth inhibition (FIG. 22B) by the drug-loaded microspheres were higher than those by the control microspheres (no drugs) (p<0.05), providing evidence of the cytotoxicity and anti-proliferative effects of the encapsulated drugs.

The stronger effects of the conjugated drugs are attributed to the conjugation of the LHRH ligand to the anticancer drugs. This is likely to increase the specificity of the binding of the released drugs to the overexpressed LHRH receptors on the MDA-MB-231 cells. Thus, the LHRH-conjugated anticancer drugs are much more effective in targeting the MDA-MB-231 cells than the unconjugated drugs.

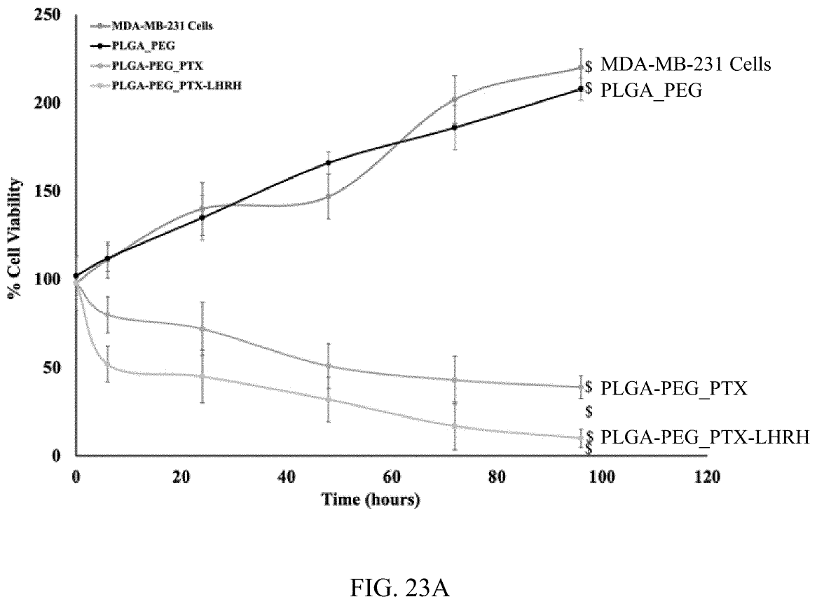

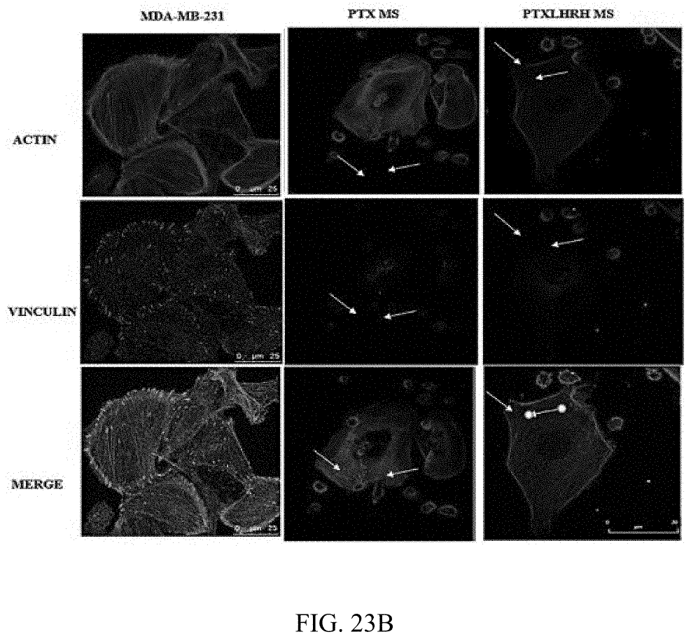

In vitro cytotoxicity and drug uptake. In this study, the cytotoxicity was considered to be a measure of the percentage of cell growth inhibition. FIG. 23A shows the extent to which the addition of the drug-loaded PLGA-PEG microspheres inhibited MDA-MB-231 cell growth after 6, 24, 48, 72 and 96 h of exposure, when compared to the inhibition of untreated cells. Higher cytotoxicity levels (due to drug-treatment) correspond to higher percentages of cell growth inhibition. The results show that cell growth was inhibited by the release of drugs from the drug-loaded PLGA-PEG microspheres (compared to control unloaded PLGA-PEG microspheres).

Furthermore, the cells treated with PLGA-PEG microspheres loaded with conjugated drugs exhibited higher percentages of cell growth inhibition than their counterparts loaded with unconjugated drugs. Hence, the LHRH-conjugated drug-loaded microspheres were more effective at inhibiting cell growth than the unconjugated drug-loaded microspheres. The increased effectiveness of the LHRH-conjugated drugs is attributed to the specific targeting of the LHRH receptors on the MDA-MB-231 cells.

Finally, the Trypan blue dye (TBD) cell count was used to confirm the effects of the drug-loaded PLGA-PEG microsphere treatment on MDA-MB-231 cell viability. An exponential increase in the cell viability/proliferation of the MDA-MB-231 cells (control) was observed throughout the incubation period. In agreement with the Alamar Blue assay results, the viability of the MDA MB 231 cells treated with PLGA-PEG microspheres (loaded with conjugated drug) were significantly reduced, in comparison to MDA-MB-231 cells treated with PLGA-PEG microspheres loaded with unconjugated drugs. This again shows that the conjugated drugs were effective at reducing cell viability than the unconjugated drugs. In summary, the TBD revealed that 95% of the cells were dead (with 5% of viable cells remaining) after 96 h of exposure to targeted encapsulated drug-loaded PLGA-PEG microspheres. The results show a significant difference between the cell viability of encapsulated conjugated drug system and unconjugated drugs since the p-value calculated is <0.05.

The network of the cytoskeleton of actin microfilaments, intermediate filaments, and microtubules make up the cytoplasm which controls the mechanical structure and shape of the cell. Hence, the disruption of the spatial organization of the cytoskeleton networks (by pharmacological treatments) can affect the structure and properties of the cell. Hence, in this section, changes in the cytoskeleton structure are elucidated following exposure to the release of cancer drugs, both conjugated and non-conjugated. The resulting effects of the uptake of cancer drugs was elucidated via confocal laser scanning microscopy and are presented in FIG. 23B. Distinctive changes in the cytoskeletal structures were observed after 5 h of exposure to drug release. The changes in the cytoskeletal structure also continue with increasing exposure to the released drugs. This result suggests that the exposure to cancer drugs significantly affects the underlying cytoskeletal structure giving rise to apoptosis and cell death.

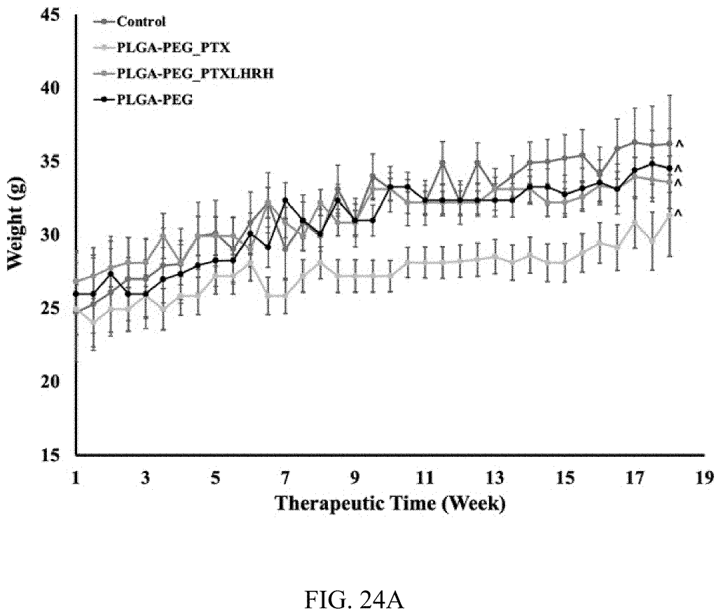

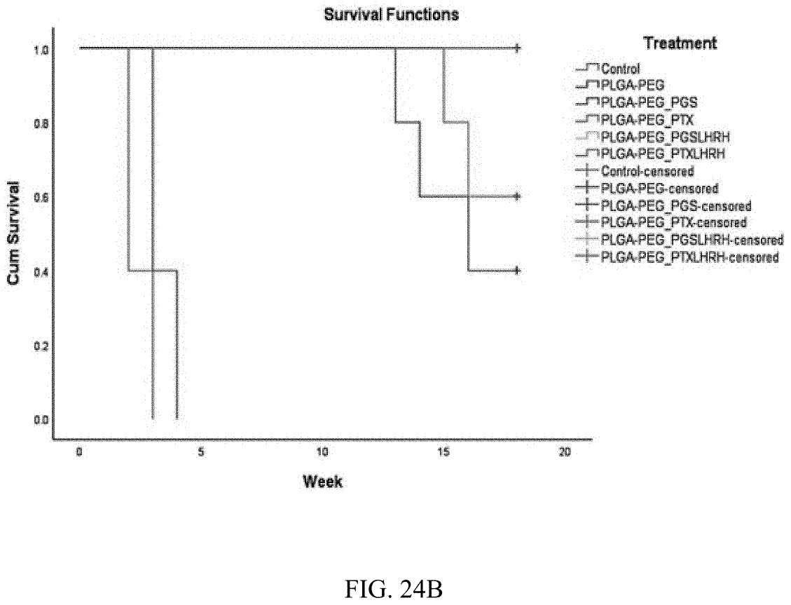

In vivo animal studies. FIG. 24A presents the body weights of the mice over the therapeutic period of 18 weeks. Results showed that there were no statistical difference in the growth rate (as a function of weight) of mice treated with drug-loaded microspheres and the control group. It can be concluded that there were no significant changes in the body weight associated with any of the treatment groups as compared to the control group. This implies that the drug-loaded particles used did not create any cytotoxic effects on the general well-being of the treatment group mice during the therapeutic window/time. Although there was an increase in body weight of the treatment groups, this increase is synonymous to those of the control group indicating that there was no noticeable side effects, physiological changes, or drastic decrease in the body weight after the administration of the drugs, compared to the control mice. Consequently, during the therapeutic time, all of the mice studied appeared to be healthy with normal eyes and skin conditions. It was found that the concentration of the conjugated drugs used are effective for the treatment of TNBC.

Survival rate for all the treatment groups during the therapeutic duration are shown is presented in the Kaplan-Meier curves as shown in FIG. 24B. A survival rate that describe the recurrence of the treated tumor was observed at week 13, 14, 16 for mice treated with unconjugated drugs, while at week 15 and 16 week a recurrence for mice treated with the conjugated drug was observed. In vivo animal studies results showed that the drug loaded microsphere prolonged the survival of mice and prevented the recurrence time for tumor. However, mice treated with targeted drug-loaded microspheres with an overlapping curve show a prolonged survival and limits recurrence compared to the unconjugated drugs. Overall, the results reveal that each group treated with drug-loaded microspheres had a higher cumulative survival compared to the cumulative survival noted in the untreated/control groups (p<0.0001). These results from are in good agreement with the in-vitro cell viability studies.

The mean tumor volume was 310±14 mm³ 28 days after the tumor was induced subcutaneously. The representative conjugated drug-loaded microspheres implanted after tumor was removed revealed that there was no local recurrent of tumor after 18 weeks. It was observed that for the case of mice implanted with conjugated drug-loaded, there was no recurrence of tumor after drug released from the microspheres for 18 weeks).

In general, for the mice treated with the conjugated drug-loaded microspheres, no significant weight loss or side effects were discussed. However, this groups implanted with positive control microspheres (PLGA-PEG) and the control mice (with no microspheres) exhibited noticeable multiple recurrences of the TNBC tumors These recurrences are attributed to the incomplete removal of all of the residual tumor and the absence of drug-loaded microspheres. In contrast, no tumor reoccurrence was observed after the implantation of the conjugated TNBC drug.

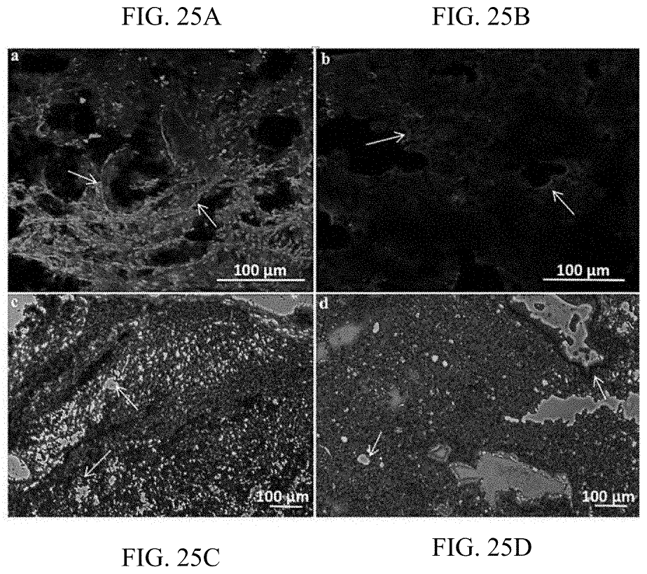

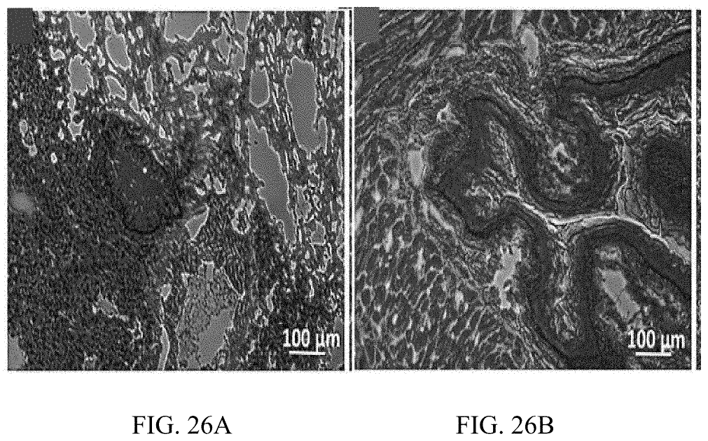

FIG. 25A and FIG. 25B present immunofluorescence (IF) images of LHRH receptors showing the presence of LHRH receptors on the tumor and lungs of the control mice group that was treated with non-drug loaded microparticles. It was also noticed that after 18 weeks of surgery, the source tumor (FIG. 25C) showed metastases in the lungs (FIG. 25D). FIG. 26A and FIG. 26B show the lungs of mice treated with unconjugated drug-loaded PLGA-PEG and conjugated drug-loaded PLGA-PEG microparticles, respectively. The results show that for the control mice, there was evidence of metastasis in the lungs, due to the presence of multiple metastatic foci or nodules from H&E histological staining. Hence, both IF staining and the H&E analyses of the primary tumors and the metastases in the lungs validated the use of conjugated drug-loaded microspheres for the localized drug delivery of LHRH-PTX to tumor sites following surgical removal of the primary tumor.

### Materials and Experimental Methods

Materials. Poly (D,L-lactide-co-glycolide) (PLGA 65:35, viscosity 0.6 dL/g), poly vinyl alcohol (PVA) (98% hydrolyzed, MW=13,000-23,000), Bovine Serum Albumin (BSA) and 4% paraformaldehyde were obtained from Sigma Aldrich (St. Louis, Mo., USA). Polyethylene glycol (PEG) (8 kD), Dichloromethane (DCM) and Phosphate Buffered Saline (PBS) solution that were used for in vitro drug release at pH of 7.4 were purchased from Fisher Scientific (Hampton, N.H., USA). Paclitaxel was obtained from ThermoFisher Scientific (Walthmam, Mass., USA) and was conjugated to LHRH.

Cell culture medium Leibovitz's-15 (L-15), trypsin-ethylenediamine-tetra-acetic acid (Trypsin-EDTA), Fetal Bovine Serum (FBS), penicillin-streptomycin, Alamar Blue Cell Viability Assay, Dulbecco's phosphate-buffered saline (DPBS), vinculin Mouse Monoclonal Antibody, Goat anti-Mouse IgG (H+L) Superclonal Secondary Antibody, Alexa Fluor 488 conjugate, Alexa Fluor 555 Rhodamine Phalloidin, Triton X-100, Trypan Blue Solution (0.4%) were also procured from ThermoFisher Scientific (Walthmam, Mass., USA). MDA-MB-231 cell line used in this study was obtained from American Type Culture Collection (ATCC) (Manassas, Va., USA). All of the reagents that were used were of analytical grade, as provided by the suppliers.

Preparation of drug-loaded PLGA-PEG microspheres. Targeted or canjgated drug-loaded microspheres (LHRH-PTX-loaded PLGA-PEG blend microspheres) and non-targeted or unconjugated drug-loaded microspheres (PTX-loaded PLGA-PEG blend microparticles) were prepared, respectively, using the emulsion solvent evaporation technique, described in prior work by Obayemi et al. Although, in this study physical blends consisting of PLGA and PEG polymer in the ratio of 1:1 were dissolved in an organic solvent (DCM) to form a primary system. In separate vials, 5 mg/ml drug concentration (PTX or LHRH-PTX) were prepared and emulsified in a 3% PVA stabilizer. These were then transferred under homogenization to the primary solution.

The resulting drug-polymer mixtures were sonicated to form a homogenous initial oil-water system. The homogeneous emulsion was then transferred dropwise into an aqueous 3% PVA solution (prepared with deionized water). The mixture formed was homogenized with an Ultra Turrax T10 basic homogenizer (Wilmington, N.C., USA) that was operated at 30,000 rpm for 5 min. The resulting oil-water emulsion was then stirred with a magnetic stirrer for 3 h to enable the evaporation of the DCM.

The excess amount of PVA in the stirred mixture was removed by washing four times with tap water and centrifuging for 10 min at 4,500 rpm with an Eppendorf Model 5,804 Centrifuge (Hauppauge, N.Y., USA). The emulsifier/stabilizer and non-incorporated drugs were then washed off, while the drug-encapsulated microparticles were recovered after centrifugation. Finally, the resulting microparticles were lyophilized for 48 h with a VirTis BenchTop Pro freeze dryer (VirTis SP Scientific, NY, USA). The lyophilized microparticles powder were stored at −20° C., prior to the material characterization and drug release experiments. PLGA-PEG microparticles (without drugs) were also prepared as controls.

Drug-loaded microparticles. The hydrodynamic diameters and polydispersity index of the lyophilized drug-loaded and control PLGA-PEG microparticles were analyzed using a Malvern Zetasizer Nano ZS (Zeta-sizer Nano ZS, Malvern Instrument, Malvern, UK). The morphologies of the microparticles were also characterized using Scanning Electron Microscopy, (SEM) (JEOL 7000F, JEOL Inc. MA, USA). Prior to SEM, the freeze-dried microparticles were mounted initially on double-sided copper tape on an aluminum stub. The resulting particles were then sputter-coated with a 5 nm thick layer of gold. The mean diameter of the microparticles were then analyzed using the ImageJ software package (National Institutes of Health, Bethesda, Md., USA).

Fourier Transform Infrared Spectroscopy (FTIR) (IRSpirit, Shimadzu Corporation, Tokyo, Japan) was used to characterize the physicochemical properties of the drug-loaded PLGA-PEG microparticles. This was used to evaluate the chemical bonds/functional groups that were associated with the drug-loaded and unloaded PLGA-PEG microparticles. The lyophilized samples were scanned at 4 mm/s at a resolution of 2 cm-1 over a wavenumber range of 600-3,600 cm⁻¹. This was done using the IR solution software package (ver.1.10) (IRSpirit, Shimadzu Corporation, Tokyo, Japan).

Nuclear Magnetic Resonance Spectroscopy (NMR) was also used to study the structure of unloaded and drug-loaded PLGA-PEG microparticles. This was done using a Bruker Advance 400 MHz (Bruker BioSpin Corporation, Billerica, Mass., USA). First, 10 mg of PLGA-PEG microparticles were dissolved in 1 ml of chloroform (CD C13). HNMR spectra of drug-loaded and control PLGA-PEG microparticles were obtained and analyzed using Bruker's TopSpin Software package (ver 3.1) (Bruker Biospin GmbH, Rheinstetten, Germany).

Finally, the thermal properties of the drug-loaded PLGA-PEG microparticles and their control were measured using Thermogravimetric Analysis (TGA) (TG 209 F1 Libra, NETZSCH, Selb, Germany) and Differential Scanning Calorimetry (DSC) (DSC 214 Polyma, NETZSCH, Selb, Germany). This was done to evaluate the possible interactions of the drugs with the polymer blends (PLGA-PEG). TGA thermograms were obtained between 25 and 900° C. with a constant heating rate of 20 K/min under nitrogen gas. This was done using alumina crucibles containing 10 mg of sample.

For the DSC analysis, 10 mg of the freeze-dried drug-loaded and control PLGA-PEG microparticles was weighed, respectively. In each case, samples were sealed in aluminum pans. They were then heated in an inert nitrogen atmosphere with a nitrogen flow rate of 20 ml/min that was subjected to a heating cycle between 20 and 250° C. with an empty reference aluminum pan. The data obtained was then analyzed by NETZSCH Proteus-7.0 software (NETZSCH, Selb, Germany). Similar procedure was followed for DSC analysis of PTX. This was used to identify the decomposition temperatures, the glass transition temperatures (T_{g}) and the melting temperatures (Tₘ), respectively.

In vitro drug release. Sixty-two-day in vitro drug release experiments were performed on PLGA-PEG microparticles that were encapsulated with PTX or LHRH-PTX. These were carried out at 37° C., 41° C. and 44° C. in an effort to study the kinetics and thermodynamics of drug release under in vitro conditions. The temperatures were chosen to correspond to the normal human body temperature (37° C.) and hyperthermic temperatures (41° C. and 44° C.).

First, triplicate 10 mg measures of drug-loaded microparticles were suspended separately in 10 ml of PBS of pH 7.4 containing 0.2% Tween 80, using 15 ml screw-capped tubes. The sample tubes were then placed in orbital shakers (Innova 44 Incubator, Console Incubator Shaker, New Brunswick, N.J., USA) rotating at 80 rpm and maintained at temperatures of 37° C., 41° C., and 44° C., respectively. At 24-h intervals, over a period of 62 days, the tubes were centrifuged at 3,000 rpm for 5 min to obtain 1.0 ml of the centrifuged supernatant (known release study samples). 1 ml of freshly prepared-drug free PBS was then used to replace the removed supernatant to conserve the sink conditions. The test samples were then swirled and placed back into the shaker incubator for the continuous release study.

The amount of released drug in each of the supernatant samples (released at 37° C., 41° C. and 44° C.) was characterized using a UV-Vis spectrophotometer (UV-1900 Shimadzu Corporation, Tokyo, Japan). The wavelength of the UV-Vis spectrophotometer was fixed at a wavelength of 229 nm (PTX and LHRH-PTX) in order to measure the absorbance. A standard curve was used to determine the concentrations of drug (PTX and LHRH-PTX) released from their respective drug-loaded microparticles.

The drug encapsulation efficiencies of the microspheres were also determined. First, 10 mg of microparticles was dissolved in DCM. The amount of drug encapsulated was then determined with a UV-Vis spectrophotometer (UV-1900 Shimadzu Corporation, Tokyo, Japan) at a fixed maximum wavelength of 229 nm for PTX and LHRH-PTX. The amount of drug that was encapsulated into the PLGA-PEG microparticles was then determined from the weight of the initial drug-loaded microparticles and the amount of drug incorporated, using a method developed by Park et al.

The Drug Loading Efficiency and Drug Encapsulation Efficiency (DEE) of drug-loaded PLGA-PEG micro-particles was determined from Eqs. (1) and (2), respectively:

$$\begin{matrix}Drug encapsulation efficency ( DLE ) = \frac{MD}{MD + MP} \times 100 & ( 1 ) \\ Drug encapsulation efficency ( DEE ) = \frac{Mx}{Mz} \times 100 & ( 2 )\end{matrix}$$

where MD is the mass of drug uptake into the microspheres, MP of polymer in the microsphere, Mₓ is the amount of encapsulated drug and Mz is the amount of drug used for the preparation of the microparticle.

Since drug release is often enabled by capsule degradation, the degradation of the drug-loaded microparticles was studied after each week of degradation under in vitro conditions. This was done using Scanning Electron Microscopy, (SEM) (JEOL 7000F, JEOL Inc. MA, USA), which was used to characterize the microstructural morphologies of the drug-loaded polymer blend.

Modeling. Kinetics modeling. The drug release kinetics of drug-loaded PLGA-EG microparticles were determined by fitting the release data to Zeroth order kinetics, First Order Kinetics, Higuchi Model and Kors-meyer-Peppas Model. Zeroth order kinetics was initially used to describes the release from the drug-loaded microspheres in which the release rate is independent of concentration. Hence, the plot of % Cumulative Drug Release (CDR) versus time was obtained based Eq. (3) below:

Qₜ=Q_{O}+K₀·t (3)

where Qₜ is the cumulative amount of drug released in time ‘t’ (release occurs rapidly after drug dissolves), Q₀ is the initial amount of drug in the solution and K₀ is the zeroth order release constant and ‘t’ is time in hours.

In the case of first order kinetics, our release rate was shown to depend on concentration. A plot of log of % cumulative drug release (CDR) versus time that gives a straight line was plotted based on Eq. (4):

log Qₜ=log Q₀+Kt/2.303 (4)

where Qₜ is the cumulative amount of drug release in time ‘t’, Q₀ is the initial amount of drug in the solution, K is the first order release constant, and ‘t’ is time. First order kinetics is often observed during the dissolution of water-soluble drugs in porous matrices.

Furthermore, the Higuchi model was used to characterize the release of the drugs incorporated into polymer matrices. Typically, the Higuchi model describes the drug release from insoluble matrix as a square root of time based on Fick's first law, 58. t A p lot of % Cumulative Drug Release (CDR) versus the square root of time (\sqrt{t}) as shown by Eq. (5) was used to describe the kinetics of drug release.

Q^{t}=K_{H}·t½ (5)

where Qₜ is the cumulative amount of drug released at time (t), K_{H} is Higuchi constant and ‘t’ is time.

Finally, the Korsmeyer-Peppas (K-P) model was also used to explore the drug release kinetics from the polymeric matrix systems. For K-P drug release, a plot of

$$log \frac{Mt}{Moo}$$

versus log t was plotted where ‘n’ represents the slope of the line, which corresponds to the underlying mechanism of drug release. The diffusion exponent (n value) of Korsmeyer-Peppas model was then used to identify the different drug release mechanism. For example, n<0.45 corresponds to a Fickian diffusion mechanism, while 0.45<n<0.89 corresponds to non-Fickian transport, n=0.89 corresponds to Case II (relaxational) transport, while n>0.89 corresponds to super case II transport. The K-P model is given by (6):

$$\frac{Mt}{Moo} = K {t}^{n} \qquad ( 6 )$$

Where

$$\frac{Mt}{Moo}$$

is a fraction of drug released after time ‘t’, ‘K’ is the kinetic constant, n is the release exponent, and ‘t’ is time. In most cases, the K-P model is only applicable to the first 60% of drug release.

Thermodynamics of in vitro drug release. The drug release studies were used to obtain the Gibbs free energy (ΔG), the enthalpy (ΔH), and the entropy (ΔS) changes associated with drug release from the drug-loaded PLGA-PEG microparticles at different temperatures. The values of ΔG, ΔH and ΔS obtained were then used to explain the thermodynamic properties and the spontaneity of the underlying drug release processes from the drug-loaded microspheres.

Initially, the experimental data obtained from our drug release experiments (at different temperatures) were used to estimate the activation energy (Eₐ). This is done using the Arrhenius Eq. (8). The underlying thermodynamical mechanisms were then elucidated from Eqs. (7) and (8). These give:

$$\begin{matrix}Kt = Dfe \frac{Ea}{RT} & ( 7 ) \\ and & \\ {ln K}_{t} = {ln D}_{f} - \frac{{E}_{a}}{R} \frac{1}{T} & ( 8 )\end{matrix}$$

where R is the universal gas constant (8.314 J mol⁻¹ K⁻¹), Kₜ is the thermodynamic equilibrium constant, T is given as the absolute temperature (K), Eₐ is the activation energy, D_{f} is the pre-exponential factor and Kₜ is the thermodynamic equilibrium constant. The activation energy, Eₐ (kJ mol), was estimated from a Van Hoff plot of ln Kₜ versus 1/T. Hence, the slope of the plot gives

$$- \frac{{E}_{a}}{R}$$

The Eyring expression for Kₜ gives (9):

$$ln \frac{{K}_{r}}{T} = - \frac{Δ H}{R} \frac{1}{T} + ln \frac{{K}_{B}}{h} + \frac{Δ S}{R} \qquad ( 9 )$$

In cases in which the plot of ln Kₜ versus 1/T is linear, then the underlying enthalpy ΔH (slope) and entropy ΔS (intercept) can be determined, respectively from the slopes and intercepts of the plots. Hence, the slope ‘m’ is given as

$$- \frac{Δ H}{R}$$

and the intercept ‘c’ is given by ln

$$\frac{K B}{h} + \frac{Δ S}{R}$$

where ΔH is the enthalpy change, ΔS is the entropy change, K_{B} is the Boltzmann constant (1.38065 m² kg s⁻² k⁻¹), and h is the Planck's constant (6.626×10⁻³⁴ J s). Finally, the changes in the free energy AG can be obtained by substituting the calculated values of ΔH and ΔS into Eq. (10) at a given temperature, T.

Finally, the Gibbs free energy change is given by (10):

ΔG=ΔH−TΔS (10)

where ΔS is the entropy change, ΔH is the enthalpy change and ΔG is Gibbs free energy change.

Cell culture experiments. The MDA-MB-231 breast cancer cells were cultured in Leibovitz's 15 (L-15) medium, supplemented with 10% FBS and penicillin/streptomycin (50 U/ml penicillin; 50 μg/ml streptomycin). This complete cell culture medium containing L-15 and other supplements (10% FBS and 2% penicillin/strep-tomycin) is referred to as L-15⁺.

In vitro cell viability and cytotoxicity. In vitro cell viability and cytotoxicity studies were performed using the Alamar Blue Cell Assay as described in our recent studies. This was used to explore the possible effects of drug-induced toxicity on triple negative breast cancer (MDA-MB-231) cells. 10⁴ cells/well were seeded in 24-well plates (n=4) in L-15⁺ culture medium. Furthermore, three hours after cell attachment, the culture medium was replaced with 1 ml of culture medium containing 0.5 mg/ml drug-loaded PLGA-PEG microparticles.

Cell viability was monitored at durations of 0, 6, 24, 48 72 and 96 h after drug-loaded microparticle addition. At each of these time points, the culture medium (L-15⁺) was replaced with 1 ml of culture medium (L-15⁺) containing 10% alamar blue solution. The resulting cells in the 24 well-plates were then incubated in a humidified incubator at 37° C. for 3 h. 100 μl aliquots were transferred into duplicate wells of a black opaque 96-well plate (Thermo Fisher Scientific, Waltham, Mass.) for fluorescence intensities measurement at 544 nm excitation and 590 nm emission using a 1420 Victor3 multilabel plate reader (Perkin Elmer, Waltham, Mass.). All of the experiments were repeated thrice.

The percentage of alamar blue reduction and the percentage of cell growth inhibition were determined from Eq. (11) and (12).

$$\begin{matrix}\% Reduction = \frac{{FI}_{sample} - {FI}_{10 \% AB}}{{FI}_{100 \% R} - {FI}_{10 \% AB}} \times 100 & ( 11 ) \\ \% Growth inhibition = ( 1 - \frac{{FI}_{sample}}{{FI}_{cells}} ) \times 100 & ( 12 )\end{matrix}$$

where Fₛₐₘₚₗₑ is the fluorescence intensity of the samples, FI_{10% AB} is the fluorescence intensity of 10% Alamar Blue reagent (negative control), FI_{100% R} is the fluorescence intensity of 100% reduced Alamar Blue (positive control) and FI_{cells} is the fluorescence intensity of untreated cells.

The loss of cell viability was characterized using a dye exclusion assay. This works based on the concept that viable cells do not take up impermeable dyes (like Trypan Blue), while dead cells are permeable and take up the dye because their membranes lose their integrity. In this work Trypan Blue Dye (TBD) staining was used to quantify the loss of cell viability. This utilized a 0.4% solution of TBD in buffered isotonic salt solution with a pH of 7.3. 0.1 ml of TBD stock solution was added to 1 ml of cells, mixed gently and incubated at 25° C. for 1 min. A hemocytometer was then used to count the number of blue staining cells, and the total number of cells under an optical microscope (Nikon TS100, Nikon Instruments Inc., Melville, N.Y., USA) that was operated at low magnification 24.

% Viable cells (VC)=1−(Number of blue cells÷Number of total cells)×100 (13)

Cellular drug uptake. MDA-MB-231 cells were seeded on coverslips (CELLTREAT Scientific Products, Pep-perell, MA, USA) in 12-well plates using 1 ml growth medium (L-15⁺). The cells were then incubated in a humidified incubator at 37° C. until cells were about 70% confluent. Post attachment, the cells were incubated with 1 ml of 0.1 mg/ml drug-loaded microspheres dissolved in growth medium (L-15⁺). After 5 h, the cells were washed twice with 5% (v/v) Dulbecco's phosphate-buffered saline (DPBS) (Washing solvent). After washing, the cells were then fixed with 4% paraformaldehyde for 12 min, before rinsing thrice with 5% (v/v) DPBS. 0.1% Triton X-100 was added for 10 min to permeabilize the cells. This was then blocked with 1% BSA for 1 h at room temperature (25° C.). The BSA-treated ECM were then rinsed thrice with the 5% (v/v) DPBS, before labeling with vinculin Mouse Monoclonal Antibody at 2 μg/ml and incubating for 3 h at room temperature (25° C.).

The washing solvent was used to rinse the resulting samples, which were then labeled with Goat anti-Mouse IgG (H+L) Superclonal Secondary Antibody, Alexa Fluor 488 conjugate for 45 min at room temperature. F-actin was stained with Alexa Fluor 555 Rhodamine Phalloidin for 30 min. The coverslips were then mounted on glass slides and sealed. The cells were visualized with HEPES buffer (pH 8) using HCX PL APO CS 40X 1.25 oil objective in Leica SP5 Point Scanning Confocal Microscope (Buffalo Grove, Ill., USA) and representative images were obtained.

In vivo studies. In vivo animal studies similar to our recent studies were carried in this work using thirty 3-week old healthy immunocompromised female athymic nude-Foxn1nu mice. These mice were purchased from Envigo (South Easton, Mass., USA) and have a weight of 16 g. These mice were kept in the vivarium (to acclimatize) until they are 4-weeks old. They were then used in in vivo studies to explore the extent to which encapsulated localized and targeted drug delivery systems can be used to prevent the breast tumor regrowth or locoregional recurrence, following surgical resection.

All the animal procedures described in this work were performed in accordance with the approved animal guidelines by the Worcester Polytechnic Institute (WPI), Institutional Animal Care and Use Committee (WPI IACUC) with approval number #A3277-01. The mice were also maintained in accordance with the approved IACUC protocol and were provided with autoclaved standard diet. All the experimental protocols in these stud¬ies were performed under an approved ethical procedure and guidelines provided by the Worcester Polytechnic Institute IACUC. The sample group are based on the agent that are implanted into the mice for the treatment. The number of mice per this sample group (n) was determined to be n=5 based on power law and from our prior work. The thirty mice were randomly divided into six groups of five mice each. Each of this group was exposed to one of the following: (PLGA-PEG-PTX, PLGA-PEG-LHRH-PTX), positive control (PLGA-PEG) and control group (without microsphere).

When the mice in each study group were 4-weeks-old, interscapular subcutaneous TNBC tumors were induced via the subcutaneous injection of 5.0×10⁶ MDA-MB-231 cells that were harvested from monolayer in vitro cell cultures. Subcutaneous tumors were allowed to grow for over 4 weeks until they were large enough to enable tumor surgery and microsphere implantation (28 days after tumor induction). The expected size of the induced subcutaneous xenograft tumor after 28 days of induction is 300 21 mm³. The tumor formation was investigated by palpation, which was measured on a daily basis with digital calipers. During this period, the mice were monitored for changes in weight, abnormalities and infections. For baseline evaluation, control mice (without microspheres) were also monitored for comparisons with the mice injected with drug-loaded microspheres.

Tumor volume was calculated from the following formula:

Tumor=a×b²/2 (14)

where a and b are the respective longest and shortest diameters of the tumors that were measured using a digital Vernier caliper.

Surgical removal of ˜90% of the tumor was performed randomly on each group member using the recommended anesthesia and pain suppressant. In each case, 200 mg/ml of PLGA-PEG-PTX, PLGA-PEG-LHRH-PTX, positive controls (PLGA-PEG) and control were implanted locally at the location where the source resected tumor was removed. The statistical rationale for each treatment group was based on power law and from our prior work. Within each group, localized cancer drug release was monitored for the period of 18 weeks. The body weight of each mice was monitored and measured every 3 days up to 126 days to check for any possible weight loss/gain, physiological changes, toxicity to the drugs, and well-being of the mice for the different treatment groups. This was done to check for possible tumor regrowth. In a similar fashion, after the 18 weeks of study, the mice were euthanized and their tumors and lungs were then excised. This was followed by cryo-preservation to check for any toxicity and metastasis.

Following weight analysis, the survival rate of the various treatment groups was compared as a function of recurrence of the TNBC tumor. Survival study of mice was done post-surgical removal of tumor and during treatment period. The mice were observed for 18 weeks post treatment for signs of cancer recurrence, if any. This was to allow enough time for recurrence. Thirty female nude mice were randomly divided into the following groups (n=4): Control, PLGA-PEG, PLGA-PEG-PTX, PLGA-PEG-PTXLHRH. Survival curves were made using Kaplan-Meier plots, and the statistical difference was evaluated using the log-rank test in SPSS. The mice in this study were euthanized when reoccurrence were observed. At the end of week 18, the surviving mice were also euthanized.

Histopathological study and immunofluorescence staining. The histopathology of the lungs, and in some cases regrowth/reoccurred tumor were evaluated. The samples that were used for the histological examination of the lungs were sectioned into 5 μm thicknesses along the longitudinal axis using similar technique from our recent studies. They were then placed on a glass slide. First, the slides were hydrated by passing them through 100, 90 and 70% of alcohol baths. The hydrated samples (on the slides) were then stained with hema-toxylin and eosin (H&E). The stained slides were finally examined using light microscopy (with a 20× objective lens) in a model TS100F Nikon microscope (Nikon Instruments Inc., Melville, N.Y., USA) that was coupled to a DS-Fi3 C mount that was attached to a Nikon camera.

Receptor staining via immunofluorescence (IF) staining was used to characterize the overexpressed LHRH receptors on the TNBC tumor and organs. This was crucial to show evidence of regrowth or the presence of metastasis in the organs using the IF staining method as described in prior work. Optimum cutting temperature (OCT) compound-Embedded frozen tumor/tissue were processed in a cryostat (Leica CM3050 S Research Cryostat, Leica Biosystems Inc., Buffalo Grove, Ill., USA). The stained samples were then imaged at a magnification of 40× in a Leica TCS SP5 Spectral Confocal microscope that was coupled to an Inverted Leica DMI 6000 CS fluorescence microscope (Leica, Buffalo Grove, Ill., USA).

Statistical analysis. The results are reported as mean standard deviation for n=3 (unless otherwise stated). In the in vitro study of drug release, cell viability studies as well as the in vivo study of the effects of drug release, statistical differences between the treatment groups were analyzed using one-way ANOVA. Differences in in vitro cell viabilities between the different treatment groups at different durations were analyzed using two-way ANOVA with post hoc Tukey HSD multiple comparisons tests using IBM SPSS Statistics 25 package. The differences were considered to be significant when the p-value was <0.05.

All patents, patent applications, and published references cited herein are hereby incorporated by reference in their entirety. It will be appreciated that several of the above-disclosed and other features and functions, or alternatives thereof, may be desirably combined into many other different systems or application. Various presently unforeseen or unanticipated alternatives, modifications, variations, or improvements therein may be subsequently made by those skilled in the art.

What is claimed is:

1. A conjugate comprising a Luteinizing Hormone Releasing Hormone (LHRH) or an analog of LHRH conjugated to paclitaxel active agent.

2. The conjugate of claim 1, wherein the analog of LHRH is D-Lys6 LHRH.

3. The conjugate of claim 1, wherein the paclitaxel active agent is conjugated at the epsilon (c) amino side chain of the LHRH or the analog of LHRH.

4. The conjugate of claim 1, further comprising a hydrophilic linker, wherein the hydrophilic linker conjugates paclitaxel active agent to the LHRH or the LHRH analog.

5. The conjugate of claim 1 wherein the linker is N-hydroxysuccinimide, 1-ethyl-3-(3-dimethylaminopropyl)carbodiimide hydrochloride (EDC) or combinations thereof.

6. A pharmaceutical composition comprising (a) an effective amount of a conjugate comprising a Luteinizing Hormone Releasing Hormone (LHRH) or an analog of LHRH conjugated to paclitaxel active agent, and (b) a physiologically acceptable carrier.

7. The pharmaceutical composition of claim 6, wherein the analog of LHRH is D-Lys6 LHRH.

8. The pharmaceutical composition of claim 6, wherein the paclitaxel active agent is conjugated at the epsilon (c) amino side chain of the LHRH or the analog of LHRH.

9. The pharmaceutical composition of claim 6, wherein the conjugate further comprises a hydrophilic linker, wherein the hydrophilic linker conjugates paclitaxel active agent to the LHRH or the LHRH analog.

10. The pharmaceutical composition of claim 9, wherein the linker is N-hydroxysuccinimide, 1-ethyl-3-(3-dimethylaminopropyl)carbodiimide hydrochloride (EDC) or combinations thereof.

11. The pharmaceutical composition of claim 6, wherein the pharmaceutical composition comprises microspheres loaded with the conjugate.

12. The pharmaceutical composition of claim 11, wherein the microspheres are poly lactic-co-glycolic acid-polyethylene glycol (PLGA_PEG) polymer microspheres.

13. The pharmaceutical composition of claim 6, wherein the pharmaceutical composition is formulated for intravenous injection.

14. A method for treating breast cancer, comprising: administering to a subject in need thereof an effective amount of a pharmaceutical composition comprising a conjugate of a Luteinizing Hormone Releasing Hormone (LHRH) or an analog of LHRH conjugated to paclitaxel active agent, and a physiologically acceptable carrier.

15. The method of claim 14, wherein the analog of LHRH is D-Lys6 LHRH and the paclitaxel active agent is conjugated at the epsilon (c) amino side chain of the D-Lys6 LHRH moiety.

16. The method of claim 14, wherein the conjugate further comprises a hydrophilic linker to conjugate the paclitaxel active agent to the LHRH analog, the linker comprising N-hydroxysuccinimide (NHS), 1-ethyl-3-(3-dimethylaminopropyl)carbodiimide hydrochloride (EDC) or combinations thereof.

17. The method of claim 14, wherein the pharmaceutical composition comprises poly lactic-co-glycolic acid-polyethylene glycol (PLGA_PEG) polymer microspheres loaded with the conjugate.

18. The method of claim 14, wherein the pharmaceutical composition is administered intravenously.

19. The method of claim 15, wherein the subject in need thereof suffers from triple negative breast cancer.

20. The method of claim 14 comprising administering the pharmaceutical composition intravenously and subsequently injecting polymer microspheres loaded with the conjugate in proximity of tumor.


SEQUENCE LISTING (3 sequences)

SEQ ID NO: 1 - protein, 10 aa, Artificial Sequence
  Description of Artificial Sequence Synthetic peptide
  MOD_RES (1)..(1): Pyroglutamic acid
EHWSYGLRPG

SEQ ID NO: 2 - protein, 10 aa, Artificial Sequence
  Description of Artificial Sequence Synthetic peptide
  MOD_RES (1)..(1): Pyroglutamic acid
  MOD_RES (6)..(6): D-tryptophyl, D-leucyl, D-alanyl, iminobenzyl-D-histidyl, 3-(2-naphthyl)-D-alanyl, O-tert-butyl-D-seryl, D-tyrosyl, D-lysyl, D-phenylalanyl, 1-benzyl-D-histidyl or N-methyl-D-alanyl
  MOD_RES (7)..(7): L-leucyl, D-leucyl, N-alpha-methyl D-leucyl, N-alpha-methyl-L-leucyl or D-alanyl
  MOD_RES (10)..(10): (Aza)glycyl or absent
  C-term NHR1, wherein R1 is H, lower alkyl or lower haloalkyl
EHWSYXXRPX

SEQ ID NO: 3 - protein, 10 aa, Artificial Sequence
  Description of Artificial Sequence Synthetic peptide
  MOD_RES (1)..(1): Pyroglutamic acid
  MOD_RES (6)..(6): D-amino acid
EHWSYKLRPG